(19) United States
(12) Patent Application Publication
(10) Pub. No.: US 2025/0326742 A1
(43) Pub. Date: Oct. 23, 2025

(54) THERAPEUTIC COMPOUNDS

(71) Applicant: RUTGERS, THE STATE UNIVERSITY OF NEW JERSEY, New Brunswick, NJ (US)

(72) Inventors: Jun Wang, New Brunswick, NJ (US); Bin Tan, New Brunswick, NJ (US); Prakash Jadhav, New Brunswick, NJ (US); Ahmadullah Ansari, New Brunswick, NJ (US); Francesc Xavier Ruiz Figueras, New Brunswick, NJ (US); Edward V. Arnold, New Brunswick, NJ (US)

(73) Assignee: RUTGERS, THE STATE UNIVERSITY OF NEW JERSEY, New Brunswick, NJ (US)

(21) Appl. No.: 19/186,188

(22) Filed: Apr. 22, 2025

Related U.S. Application Data
(60) Provisional application No. 63/637,772, filed on Apr. 23, 2024.

(51) Int. Cl. C07D 403/14; A61K 9/00; A61K 9/12; A61K 9/20; A61K 9/48; A61K 31/4155; C07D 231/12

(52) U.S. Cl. CPC C07D 403/14; A61K 9/0019; A61K 9/12; A61K 9/2054; A61K 9/4825; A61K 31/4155; C07D 231/12

(57) ABSTRACT

The invention provides a compound of formula (I):

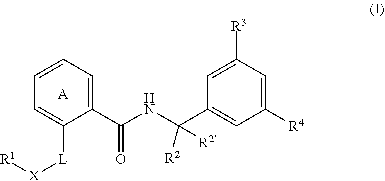

or a salt thereof, R¹-R⁴, X, L, and ring A have any of the values described in the specification, as well as compositions comprising a compound of formula (I) or a pharmaceutically acceptable salt thereof. The compounds are useful as inhibitors of papain-like protease and as antiviral agents.

### CROSS-REFERENCE TO RELATED APPLICATION

This application claims priority to U.S. Provisional Application No. 63/637,772 that was filed on Apr. 23, 2024. The entire content of the applications referenced above is hereby incorporated by reference herein.

### BACKGROUND

The COVID-19 pandemic is by far the worst pandemic since the 1918 Spanish flu. The etiological agent of COVID-19 is SARS-CoV-2, a single-stranded positive sense RNA virus that belongs to the beta coronavirus genus (Hu, B., et al., Nat Rev Microbiol 2021, 19, 141-154). Two additional coronaviruses within the same genus, SARS-CoV, and MERS-CoV, have caused epidemics in humans with mortality rates of 9.6% and 34.3%, respectively. Although SARS-CoV-2 has a lower mortality rate of 2.1% compared to SARS-CoV and MERS-CoV, it has led to a far greater death toll due to its higher transmission (Piroth, L, et al., The Lancet Respiratory Medicine 2021, 9, 251-259). SARS-CoV-2 differs from SARS-CoV and MERS-CoV in that it has a long incubation time after the initial infection (1 to 2 weeks), and a large percentage of infected patients continue to shed the virus while being asymptomatic, presenting a daunting task for surveillance and containment (Bar-On, Y. M., et al., Elife 2020, 9, e57309).

Three mRNA vaccines developed by Pfizer/BioNtech, Moderna, and Johnson and Johnson have been approved by FDA in the United States (Li, Y., et al., A Comprehensive Review of the Global Efforts on COVID-19 Vaccine Development. ACS Central Science 2021). For small molecule antivirals, remdesivir received FDA approval on Oct. 22, 2020 (Eastman, R. T., et al., ACS Central Science 2020, 6, 672-683). Although the polymerase of SARS-CoV-2 has proof-reading function, it continues to mutate at a rate about 10⁻⁶ per site per cycle (Bar-On, Y. M., et al., Elife 2020, 9, e57309). Several variants have already emerged and widely circulated among humans since the beginning of the pandemic (Lauring, A. S.; Hodcroft, E. B., JAMA 2021, 325, 529-531). Therefore, there is a dire need for additional antivirals with a novel mechanism of action.

Antivirals are not substituents of vaccines, but rather an important complement that can be used for the treatment of infection from both wild-type (WT) and variant viruses. Among the viral proteins that have been actively pursued as SARS-CoV-2 antiviral drug targets, the main protease (M^{pro}) and papain-like protease (PL^{pro}) are the most promising ones (Ma, C., et al., Cell Res 2020, 30, 678-692; and Sacco, M. D, et al., Sci Adv 2020, 6, eabe0751). M^{pro} and PL^{pro} are involved in the proteolytic digestion of the viral polyproteins pp1a and pp1ab, yielding individual functional viral proteins for the replication complex formation. PL^{pro} cleaves at three sites with the recognition sequence “LXGG↓XX” (Rut, W., et al., Sci Adv 2020, 6). PL^{pro} has been shown to play additional roles in dysregulating host immune response and impairing the host type I interferon antiviral effect through its deubiquitinating and deISG15ylating (interferon-induced gene 15) activities, respectively (Freitas, B. T., et al., ACS Infect Dis 2020, 6, 2099-2109; Shin, D., et al., Nature 2020, 587, 657-662; and Klemm, T., et al., Embo J 2020, 39, e106275). SARS-CoV-2 PL^{pro} cleaves ISG15 and polyubiquitin modifications from cellular proteins, and inhibition of PL^{pro} led to the accumulation of ISG15-conjugates and poly-ubiquitin-conjugates (Fu, Z., et al., Nat Commun 2021, 12, 488). While SARS-CoV PL^{pro} prefers ubiquitinated substrates, SARS-CoV-2 PL^{pro} prefers the ISGlyated proteins as substrates (Freitas, B. T., et al., ACS Infect Dis 2020, 6, 2099-2109; Shin, D., et al., Nature 2020, 587, 657-662; and Klemm, T., et al., Embo J 2020, 39, e106275). PL^{pro} is part of a membrane anchored multi-domain protein named non-structural protein 3 (nsp-3), an essential component of the replicase-transcriptase complex. The pleiotropic roles of SARS-CoV-2 PL^{pro} make it a promising antiviral drug target. Substantial morbidity and mortality associated with COVID-19 infection is caused by cytokine storm (Berlin, D. A.; Gulick, R. M.; Martinez, F. J., N Engl J Med 2020, 383, 2451-2460), and suppressing host immune response using dexamethasone and baricitinib has been shown to provide therapeutic benefits in the treatment of severe infections (Kalil, A. C., et al., New England Journal of Medicine 2020, 384, 795-807; and New England Journal of Medicine 2020, 384, 693-704).

Significant progress has been made in developing SARS-CoV-2 M^{pro} inhibitors (Ma, C., et al., Cell Res 2020, 30, 678-692; Sacco, M. D, et al., Sci Adv 2020, 6, eabe0751; Qiao, J., et al., Science 2021, 371, 1374-1378; Zhang, L., et al., Science 2020, 368, 409-412;) and the Pfizer's nirmatrelvir is approved by FDA. In comparison, PL^{pro} represents a more challenging drug target, and GRL0617 remains one of the most potent PL^{pro} inhibitors reported to date despite several high-throughput screening and medicinal chemistry optimization campaigns (Rut, W., et al., Sci Adv 2020, 6; Freitas, B. T., et al., ACS Infect Dis 2020, 6, 2099-2109; Shin, D., et al., Nature 2020, 587, 657-66; Klemm, T., et al., Embo J 2020, 39, e106275; Osipiuk, J., et al., Nat Commun 2021, 12, 743; and Ratia, K., et al., Proc. Natl. Acad. Sci. U.S.A. 2008, 105, 16119-24). GRL0617 was originally developed as a deubiquitinase inhibitor and was later identified as a SARS-CoV PL^{pro} inhibitor through a high-throughput screening (Ratia, K., et al., Proc. Natl. Acad. Sci. U.S.A. 2008, 105, 16119-24). As SARS-CoV-2 and SARS-CoV PL^{pro} share a sequence identity of 83% and similarity of 90%, GRL0617 was also repurposed for SARS-CoV-2 PL^{pro} and it was reported to inhibit SARS-CoV-2 PL^{pro} with IC₅₀ values of around 2 μM and SARS-CoV-2 viral replication with EC₅₀ values around 20 μM from multiple studies (Klemm, T., et al., Embo J 2020, 39, e106275; Fu, Z., et al., Nat Commun 2021, 12, 488; Osipiuk, J., et al., Nat Commun 2021, 12, 743; and Gao, X., et al., Acta Pharm Sin B 2021, 11, 237-245). Currently there is a need for SARS-CoV-2 antiviral agents that can be used alone or in combination with other drugs (e.g., protease inhibitors).

### SUMMARY

The invention provides SARS-CoV-2 antiviral agents that can be used alone or in combination with other drugs (e.g., antiviral agents, such as, for example, protease inhibitors).

Accordingly, in one embodiment, the invention provides a compound of the invention, which is a compound of formula (I):

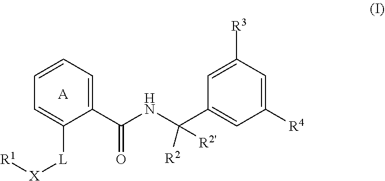

or a salt thereof, wherein:
R¹ is (C₁-C₆)alkyl, (C₂-C₆)alkenyl, (C₂-C₆)alkynyl, or —NR^{a}R^{b}, wherein any (C₁-C₆)alkyl, (C₂-C₆)alkenyl, and (C₂-C₆)alkynyl is optionally substituted with one or more groups independently selected from the group consisting of hydroxy, halo, cyano, oxo (═O), (C₁-C₆)alkoxy, —NR^{a}R^{b}, —C(═O)NR^{s}R^{t}, —C(═O)C(═O)NR^{s}R^{t}, S(═O)ₙR^{e}, and (C₃-C₆)cycloalkyl;
L is (C₁-C₃)alkylene that is optionally substituted with one or more groups independently selected from fluoro and chloro;
ring A is optionally substituted with one or more groups independently selected from the group consisting of halo, hydroxy, cyano, carboxy, (C₁-C₆)alkyl, (C₁-C₆)alkoxy, (C₁-C₆)alkoxycarbonyl, and (C₁-C₆)alkanoyloxy, wherein any (C₁-C₆)alkyl, (C₁-C₆)alkoxy, (C₁-C₆)alkoxycarbonyl, and (C₁-C₆)alkanoyloxy is optionally substituted with one or more groups independently selected from the group consisting of halo;
R² is H or (C₁-C₃)alkyl that is optionally substituted with one or more groups independently selected from halo and deuterium; and R^{2′} is H or (C₁-C₃)alkyl that is optionally substituted with one or more groups independently selected from halo and deuterium; or R² and R^{2′} taken together with the carbon to which they are attached form a (C₃-C₆)cycloalkyl;
R³ is a 5-membered heteroaryl that is optionally substituted with one or more groups independently selected from the group consisting of halo, hydroxy, cyano, (C₁-C₆)alkyl, and (C₁-C₆)alkoxy, wherein any (C₁-C₆)alkyl, and (C₁-C₆)alkoxy is optionally substituted with one or more groups independently selected from the group consisting of halo; and
R⁴ is a 5-membered heteroaryl that is optionally substituted with one or more groups independently selected from the group consisting of halo, hydroxy, cyano, (C₁-C₆)alkyl, and (C₁-C₆)alkoxy, wherein any (C₁-C₆)alkyl, and (C₁-C₆)alkoxy is optionally substituted with one or more groups independently selected from the group consisting of halo;
X is —NH—C(═O)— or —C(═O)—NH—;
R^{a} is H or (C₁-C₆)alkyl;
R^{b} is (C₁-C₆)alkyl, (C₂-C₆)alkenyl, (C₂-C₆)alkynyl, —C(═O)R^{c}, or S(═O)ₙR^{d}, wherein any (C₁-C₆)alkyl, (C₂-C₆)alkenyl, and (C₂-C₆)alkynyl is optionally substituted with one or more groups independently selected from the group consisting of halo, hydroxy, cyano, (C₁-C₆)alkoxy, —NR^{u}R^{v}, and (C₃-C₆)cycloalkyl;

R^{c} is H, (C₁-C₆)alkyl, (C₂-C₆)alkenyl, (C₂-C₆)alkynyl, 3-6 membered heterocycle, (C₁-C₆)alkanoyl, or (C₃-C₆)cycloalkyl, wherein any (C₁-C₆)alkyl, (C₂-C₆)alkenyl, (C₂-C₆)alkynyl, 3-6 membered heterocycle, (C₁-C₆)alkanoyl, and (C₃-C₆)cycloalkyl is optionally substituted with one or more groups independently selected from the group consisting of halo, hydroxy, cyano, (C₁-C₆)alkoxy, (C₁-C₆)alkoxycarbonyl, (C₁-C₆)alkanoyloxy, (C₃-C₆)cycloalkyl, oxo (═O), carboxy, —NR^{w}R^{x}, —C(═O)NR^{w}R^{x}, and (C₃-C₆)cycloalkyl;
R^{d} is (C₁-C₆)alkyl, (C₂-C₆)alkenyl, (C₂-C₆)alkynyl, or (C₃-C₆)cycloalkyl, wherein any (C₁-C₆)alkyl, (C₂-C₆)alkenyl, (C₂-C₆)alkynyl, and (C₃-C₆)cycloalkyl is optionally substituted with one or more groups independently selected from the group consisting of hydroxy, halo, cyano, (C₁-C₆)alkoxy, (C₁-C₆)alkoxycarbonyl, (C₁-C₆)alkanoyloxy, oxo (═O), NR^{y}R^{z}, carboxy, —C(═O)NR^{y}R^{z}, and (C₃-C₆)cycloalkyl;
R^{e} is (C₁-C₆)alkyl, (C₂-C₆)alkenyl, or (C₂-C₆)alkynyl, wherein any (C₁-C₆)alkyl, (C₂-C₆)alkenyl, and (C₂-C₆)alkynyl is optionally substituted with one or more groups independently selected from the group consisting of hydroxy, halo, cyano, (C₁-C₆)alkoxy, (C₁-C₆)alkoxycarbonyl, (C₁-C₆)alkanoyloxy, —NR^{y}R^{z}, carboxy, —C(═O)NR^{y}R^{z}, and (C₃-C₆)cycloalkyl;
each R^{s} and R^{t} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl, wherein any (C₁-C₆)alkyl, (C₃-C₆)cycloalkyl, (C₃-C₆)cycloalkyl(C₁-C₆)alkyl, and (C₁-C₆)alkanolyl is optionally substituted with one or more groups independently selected from (C₁-C₆)alkyl and halo; or R^{s} and R^{t} together with the nitrogen to which they are attached form a aziridino, azetidino, morpholino, piperazino, pyrrolidino or piperidino, which a aziridino, azetidino, morpholino, piperazino, pyrrolidino and piperidino,is optionally substituted with one or more groups independently selected from halo and (C₁-C₆)alkyl that is optionally substituted with one or more groups independently selected from halo;
each R^{u} and R^{v} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl, wherein any (C₁-C₆)alkyl, (C₃-C₆)cycloalkyl, (C₃-C₆)cycloalkyl(C₁-C₆)alkyl, and (C₁-C₆)alkanolyl is optionally substituted with one or more groups independently selected from (C₁-C₆)alkyl and halo; or R^{u} and R^{v} together with the nitrogen to which they are attached form a aziridino, azetidino, morpholino, piperazino, pyrrolidino or piperidino, which a aziridino, azetidino, morpholino, piperazino, pyrrolidino and piperidino,is optionally substituted with one or more groups independently selected from halo and (C₁-C₆)alkyl that is optionally substituted with one or more groups independently selected from halo;
each R^{w} and R^{x} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl, wherein any (C₁-C₆)alkyl, (C₃-C₆)cycloalkyl, (C₃-C₆)cycloalkyl(C₁-C₆)alkyl, and (C₁-C₆)alkanolyl is optionally substituted with one or more groups independently selected from (C₁-C₆)alkyl and halo; or R^{w} and R^{x} together with the nitrogen to which they are attached form a aziridino, azetidino, morpholino, piperazino, pyrrolidino or piperidino, which a aziridino, azetidino, morpholino, piperazino, pyrrolidino and piperidino,is optionally substituted with one or more groups independently selected from halo and (C₁-C₆)alkyl that is optionally substituted with one or more groups independently selected from halo;
each R^{y} and R^{z} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl, wherein any (C₁-C₆)alkyl, (C₃-C₆)cycloalkyl, (C₃-C₆)cycloalkyl(C₁-C₆)alkyl, and (C₁-C₆)alkanolyl is optionally substituted with one or more groups independently selected from (C₁-C₆)alkyl and halo; or R^{y} and R^{z} together with the nitrogen to which they are attached form a aziridino, azetidino, morpholino, piperazino, pyrrolidino or piperidino, which a aziridino, azetidino, morpholino, piperazino, pyrrolidino and piperidino,is optionally substituted with one or more groups independently selected from halo and (C₁-C₆)alkyl that is optionally substituted with one or more groups independently selected from halo; and
n is 0, 1, or 2.

The invention also provides a pharmaceutical composition comprising a compound of formula (I) or a pharmaceutically acceptable salt thereof and a pharmaceutically acceptable excipient.

The invention also provides a method for promoting an antiviral effect in an animal, comprising administering a compound of formula (I) or a pharmaceutically acceptable salt thereof to the animal.

The invention also provides a method for inhibiting a papain-like protease in an animal in need thereof, comprising administering a compound of formula (I) or a pharmaceutically acceptable salt thereof to the animal.

The invention also provides a compound of formula (I) or a pharmaceutically acceptable salt thereof for use in medical therapy.

The invention also provides a compound of formula (I) or a pharmaceutically acceptable salt thereof for the prophylactic or therapeutic treatment of a viral infection.

The invention also provides the use of a compound of formula (I) or a pharmaceutically acceptable salt thereof to prepare a medicament for treating a viral infection in an animal.

The invention also provides processes and intermediates disclosed herein that are useful for preparing a compound of formula (I) or a salt thereof.

### DETAILED DESCRIPTION

The following definitions are used, unless otherwise described: halo or halogen is fluoro, chloro, bromo, or iodo. Alkyl, alkoxy, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and and higher homologs and isomers.

The term “alkoxy” refers to an alkyl groups attached to the remainder of the molecule via an oxygen atom (“oxy”).

The term “cycloalkyl” refers to a saturated or partially unsaturated (non-aromatic) all carbon ring having 3 to 8 carbon atoms (i.e., (C₃-C₈)carbocycle). The term also includes multiple condensed, saturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane. In one embodiment the term heterocycle includes a 3-8 membered monocyclic or bicyclic heterocycle comprising 1 to 3 nitrogen atoms. In one embodiment the term heterocycle includes a 3-8 membered monocyclic or bicyclic heterocycle comprising 1 to 2 nitrogen atoms. In one embodiment the term heterocycle includes a 3-8 membered monocyclic or bicyclic heterocycle comprising 1 nitrogen atom.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl. In one embodiment the heteroaryl is a 5-membered heteroaryl. In one embodiment the heteroaryl is a 6-membered heteroaryl. In one embodiment the heteroaryl is a 5-membered heteroaryl that comprises one or two heteroatoms selected from O, N, and S. In one embodiment the heteroaryl is a 6-membered heteroaryl that comprises one or two heteroatoms selected from O, N, and S.

The term “alkoxycarbonyl” as used herein refers to a group (alkyl)-O—C(═O)—, wherein the term alkyl has the meaning defined herein.

The term “alkanoyloxy” as used herein refers to a group (alkyl)-C(═O)—O—, wherein the term alkyl has the meaning defined herein.

As used herein, the term “heteroatom” includes oxygen (O), nitrogen (N), sulfur(S) and silicon (Si).

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein a wavy line “ [symbol] ” that intersects a bond in a chemical structure indicates the point of attachment of the bond that the wavy bond intersects in the chemical structure to the remainder of a molecule.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” includes mammals.

The term “mammal” as used herein refers to humans, higher non-human primates, rodents, domestic, cows, horses, pigs, sheep, dogs and cats. In one embodiment, the mammal is a human.

The term “patient” as used herein refers to any animal including mammals. In one embodiment, the patient is a mammalian patientIn one embodiment, the patient is a human patient. In one embodiment, the mammal is a human.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; and5-membered heteroaryl can be furyl, imidazolyl, triazolyl, oxazoyl, isoxazoyl, thiazolyl, isothiazoyl, pyrazolyl, pyrrolyl, tetrazolyl, or thienyl.

A specific compound or salt is a compound of formula (I) or a salt thereof, wherein:
R¹ is (C₁-C₆)alkyl, (C₂-C₆)alkenyl, (C₂-C₆)alkynyl, or —NR^{a}R^{b}, wherein any (C₁-C₆)alkyl, (C₂-C₆)alkenyl, and (C₂-C₆)alkynyl is optionally substituted with one or more groups independently selected from the group consisting of hydroxy, halo, cyano, (C₁-C₆)alkoxy, —NR^{a}R^{b}, —C(═O)NR^{s}R^{t}, —C(═O)C(═O)NR^{s}R^{t}, S(═O)ₙR^{e}, and (C₃-C₆)cycloalkyl;
L is (C₁-C₃)alkylene that is optionally substituted with one or more groups independently selected from fluoro and chloro;
ring A is optionally substituted with one or more groups independently selected from the group consisting of halo, hydroxy, cyano, carboxy, (C₁-C₆)alkyl, (C₁-C₆)alkoxy, (C₁-C₆)alkoxycarbonyl, and (C₁-C₆)alkanoyloxy, wherein any (C₁-C₆)alkyl, (C₁-C₆)alkoxy, (C₁-C₆)alkoxycarbonyl, and (C₁-C₆)alkanoyloxy is optionally substituted with one or more groups independently selected from the group consisting of halo;
R² is H or (C₁-C₃)alkyl that is optionally substituted with one or more groups independently selected from halo and deuterium; and R^{2′} is H or (C₁-C₃)alkyl that is optionally substituted with one or more groups independently selected from halo and deuterium; or R² and R^{2′} taken together with the carbon to which they are attached form a (C₃-C₆)cycloalkyl;
R³ is a 5-membered heteroaryl that is optionally substituted with one or more groups independently selected from the group consisting of halo, hydroxy, cyano, (C₁-C₆)alkyl, and (C₁-C₆)alkoxy, wherein any (C₁-C₆)alkyl, and (C₁-C₆)alkoxy is optionally substituted with one or more groups independently selected from the group consisting of halo; and
R⁴ is a 5-membered heteroaryl that is optionally substituted with one or more groups independently selected from the group consisting of halo, hydroxy, cyano, (C₁-C₆)alkyl, and (C₁-C₆)alkoxy, wherein any (C₁-C₆)alkyl, and (C₁-C₆)alkoxy is optionally substituted with one or more groups independently selected from the group consisting of halo;
X is —NH—C(═O)— or —C(═O)—NH—;
R^{a} is H or (C₁-C₆)alkyl;
R^{b} is (C₁-C₆)alkyl, (C₂-C₆)alkenyl, (C₂-C₆)alkynyl, —C(═O)R^{c}, or S(═O)ₙR^{d}, wherein any (C₁-C₆)alkyl, (C₂-C₆)alkenyl, and (C₂-C₆)alkynyl is optionally substituted with one or more groups independently selected from the group consisting of halo, hydroxy, cyano, (C₁-C₆)alkoxy, —NR^{u}R^{v}, and (C₃-C₆)cycloalkyl;
R^{c} is H, (C₁-C₆)alkyl, (C₂-C₆)alkenyl, (C₂-C₆)alkynyl, 3-6 membered heterocycle, (C₁-C₆)alkanoyl, or (C₃-C₆)cycloalkyl, wherein any (C₁-C₆)alkyl, (C₂-C₆)alkenyl, (C₂-C₆)alkynyl, 3-6 membered heterocycle, (C₁-C₆)alkanoyl, and (C₃-C₆)cycloalkyl is optionally substituted with one or more groups independently selected from the group consisting of halo, hydroxy, cyano, (C₁-C₆)alkoxy, (C₁-C₆)alkoxycarbonyl, (C₁-C₆)alkanoyloxy, (C₃-C₆)cycloalkyl, oxo (═O), carboxy, —NR^{w}R^{x}, —C(═O)NR^{w}R^{x}, and (C₃-C₆)cycloalkyl;
R^{d} is (C₁-C₆)alkyl, (C₂-C₆)alkenyl, (C₂-C₆)alkynyl, or (C₃-C₆)cycloalkyl, wherein any (C₁-C₆)alkyl, (C₂-C₆)alkenyl, (C₂-C₆)alkynyl, and (C₃-C₆)cycloalkyl is optionally substituted with one or more groups independently selected from the group consisting of hydroxy, halo, cyano, (C₁-C₆)alkoxy, (C₁-C₆)alkoxycarbonyl, (C₁-C₆)alkanoyloxy, oxo (═O), —NR^{y}R^{z}, carboxy, —C(═O)NR^{y}R^{z}, and (C₃-C₆)cycloalkyl;
R^{e} is (C₁-C₆)alkyl, (C₂-C₆)alkenyl, or (C₂-C₆)alkynyl, wherein any (C₁-C₆)alkyl, (C₂-C₆)alkenyl, and (C₂-C₆)alkynyl is optionally substituted with one or more groups independently selected from the group consisting of hydroxy, halo, cyano, (C₁-C₆)alkoxy, (C₁-C₆)alkoxycarbonyl, (C₁-C₆)alkanoyloxy, —NR^{y}R^{z}, carboxy, —C(═O)NR^{y}R^{z}, and (C₃-C₆)cycloalkyl;
each R^{s} and R^{t} is independently selected from the group consisting of H, (C₁-C₆)alkyl, (C₃-C₆)cycloalkyl, (C₃-C₆)cycloalkyl (C₁-C₆)alkyl, and (C₁-C₆)alkanolyl, wherein any (C₁-C₆)alkyl, (C₃-C₆)cycloalkyl, (C₃-C₆)cycloalkyl (C₁-C₆)alkyl, and (C₁-C₆)alkanolyl is optionally substituted with one or more groups independently selected from (C₁-C₆)alkyl and halo; or R^{s} and R^{t} together with the nitrogen to which they are attached form a aziridino, azetidino, morpholino, piperazino, pyrrolidino or piperidino, which a aziridino, azetidino, morpholino, piperazino, pyrrolidino and piperidino,is optionally substituted with one or more groups independently selected from halo and (C₁-C₆)alkyl that is optionally substituted with one or more groups independently selected from halo;
each R^{u} and R^{v} is independently selected from the group consisting of H, (C₁-C₆)alkyl, (C₃-C₆)cycloalkyl, (C₃-C₆)cycloalkyl (C₁-C₆)alkyl, and (C₁-C₆)alkanolyl, wherein any (C₁-C₆)alkyl, (C₃-C₆)cycloalkyl, (C₃-C₆)cycloalkyl (C₁-C₆)alkyl, and (C₁-C₆)alkanolyl is optionally substituted with one or more groups independently selected from (C₁-C₆)alkyl and halo; or R^{u} and R^{v} together with the nitrogen to which they are attached form a aziridino, azetidino, morpholino, piperazino, pyrrolidino or piperidino, which a aziridino, azetidino, morpholino, piperazino, pyrrolidino and piperidino,is optionally substituted with one or more groups independently selected from halo and (C₁-C₆)alkyl that is optionally substituted with one or more groups independently selected from halo;
each R^{w} and R^{x} is independently selected from the group consisting of H, (C₁-C₆)alkyl, (C₃-C₆)cycloalkyl, (C₃-C₆)cycloalkyl(C₋₁-C₆)alkyl, and (C₁-C₆)alkanolyl, wherein any (C₁-C₆)alkyl, (C₃-C₆)cycloalkyl, (C₃-C₆)cycloalkyl(C₁-C₆)alkyl, and (C₁-C₆)alkanolyl is optionally substituted with one or more groups independently selected from (C₁-C₆)alkyl and halo; or R^{w} and R^{x} together with the nitrogen to which they are attached form a aziridino, azetidino, morpholino, piperazino, pyrrolidino or piperidino, which a aziridino, azetidino, morpholino, piperazino, pyrrolidino and piperidino,is optionally substituted with one or more groups independently selected from halo and (C₁-C₆)alkyl that is optionally substituted with one or more groups independently selected from halo;
each R^{y} and R^{z} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl, wherein any (C₁-C₆)alkyl, (C₃-C₆)cycloalkyl, (C₃-C₆)cycloalkyl(C₁-C₆)alkyl, and (C₁-C₆)alkanolyl is optionally substituted with one or more groups independently selected from (C₁-C₆)alkyl and halo; or R^{y} and R^{z} together with the nitrogen to which they are attached form a aziridino, azetidino, morpholino, piperazino, pyrrolidino or piperidino, which a aziridino, azetidino, morpholino, piperazino, pyrrolidino and piperidino,is optionally substituted with one or more groups independently selected from halo and (C₁-C₆)alkyl that is optionally substituted with one or more groups independently selected from halo; and
n is 0, 1, or 2.

A specific value for R¹ is (C₁-C₆)alkyl, (C₂-C₆)alkenyl, or (C₂-C₆)alkynyl, wherein any (C₁-C₆)alkyl, (C₂-C₆)alkenyl, and (C₂-C₆)alkynyl is optionally substituted with one or more groups independently selected from the group consisting of hydroxy, halo, cyano, (C₁-C₆)alkoxy, and (C₃-C₆)cycloalkyl.

A specific value for R¹ is —NR^{a}R^{b}.

A specific value for R¹ is selected from the group consisting of: 2-cyanoethylamino, 3-propynylamino, 2-2-dichloroethanoylamino, 2-propeneoylamino, 4-(N,N-diethylamino)2-butenoylamino, vinylsulfonylamino, acetylamino, 3-(tert-butoxycarbonyl)-2-propenoylamino, 3-(3,3-dimethylazetidinocarbonyl)-2-propenoylamino, and 3-(ethoxycarbonyl)-2-propenoylamino.

A specific value for R¹ is selected from the group consisting of:

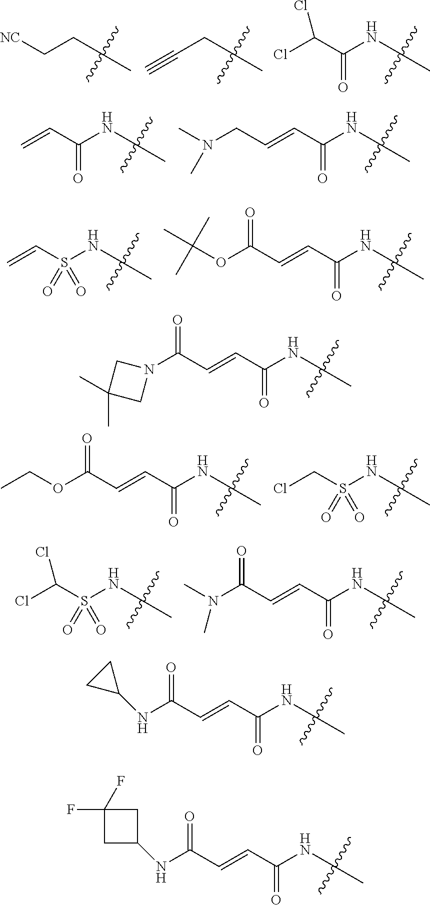

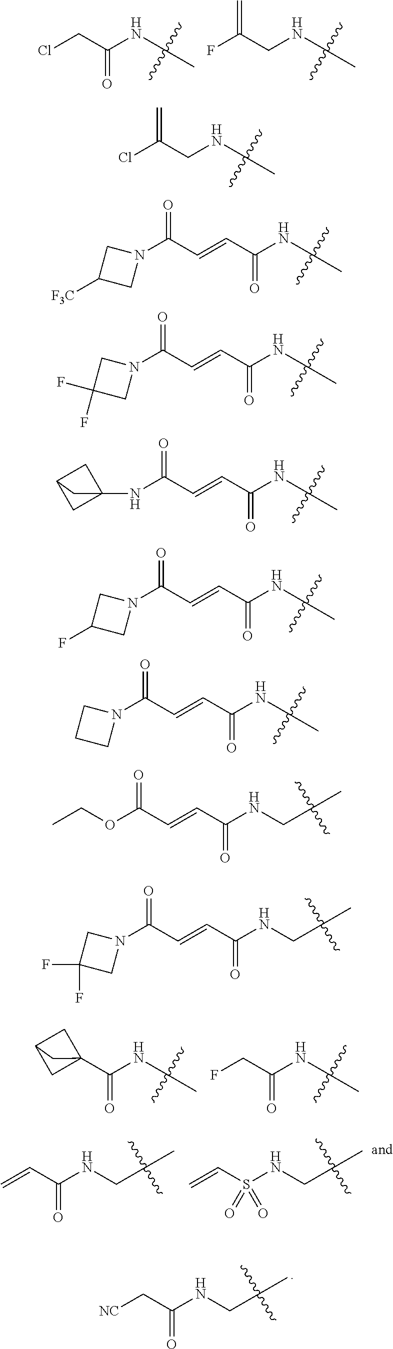

A specific value for R¹ is selected from the group consisting of:

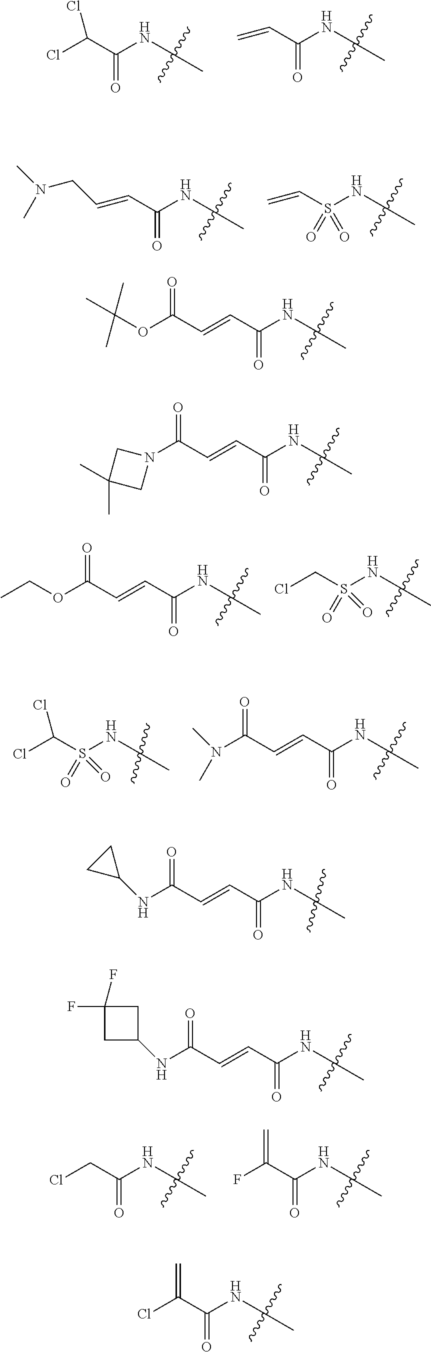

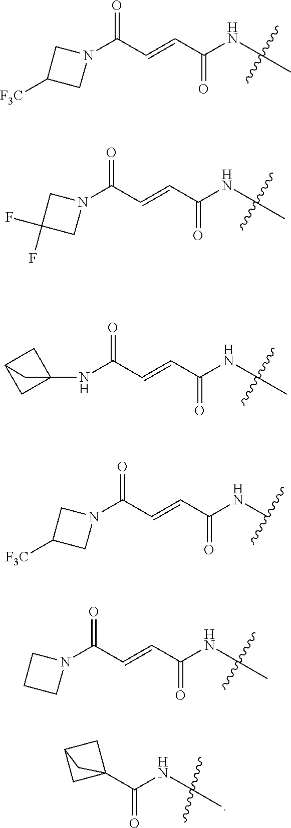 [SMILES: *NC(=O)/C=C/C(=O)N1CC(C(F)(F)F)C1 ; *NC(=O)/C=C/C(=O)N1CC(F)(F)C1 ; *NC(=O)/C=C/C(=O)NC12CC(C1)C2 ; *NC(=O)/C=C/C(=O)N1CC(C(F)(F)F)C1 ; *NC(=O)/C=C/C(=O)N1CCC1 ; *NC(=O)C12CC(C1)C2]

A specific value for R¹ is selected from the group consisting of:

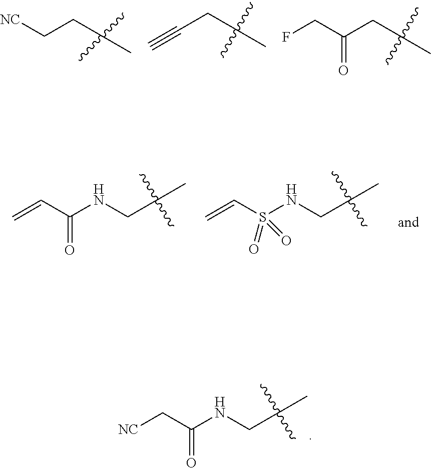

A specific value for L is (C₂)alkylene that is optionally substituted with one or more groups independently selected from fluoro and chloro.

A specific value for L is —CH₂CH₂—.

A specific value for X is —NH—C(═O)—.

A specific value for X is —C(═O)—NH—.

A specific value for R² is methyl and for R^{2′} is H.

A specific value for R³ is a 5-membered heteroaryl that comprises two nitrogen atoms that is optionally substituted with one or more groups independently selected from the group consisting of halo, hydroxy, cyano, (C₁-C₆)alkyl, and (C₁-C₆)alkoxy, wherein any (C₁-C₆)alkyl, and (C₁-C₆)alkoxy is optionally substituted with one or more groups independently selected from the group consisting of halo.

A specific value for R³ is a 5-membered heteroaryl that comprises two nitrogen atoms that is optionally substituted with one or more groups independently selected from the group consisting of (C₁-C₆)alkyl.

A specific value for R³ is:

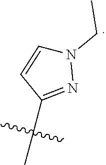 [SMILES: *c1ccn(CC)n1]

A specific value for R⁴ is a 5-membered heteroaryl that comprises two nitrogen atoms that is optionally substituted with one or more groups independently selected from the group consisting of halo, hydroxy, cyano, (C₁-C₆)alkyl, and (C₁-C₆)alkoxy, wherein any (C₁-C₆)alkyl, and (C₁-C₆)alkoxy is optionally substituted with one or more groups independently selected from the group consisting of halo.

A specific value for R⁴ is a 5-membered heteroaryl that comprises two nitrogen atoms that is optionally substituted with one or more groups independently selected from the group consisting of (C₁-C₆)alkyl.

A specific value for R⁴ is:

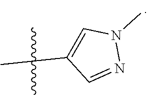 [SMILES: *c1cnn(C)c1]

A specific value for R^{a} is H.

A specific value for R^{b} is (C₁-C₆)alkyl, (C₂-C₆)alkenyl, or (C₂-C₆)alkynyl, wherein any (C₁-C₆)alkyl, (C₂-C₆)alkenyl, and (C₂-C₆)alkynyl is optionally substituted with one or more groups independently selected from the group consisting of halo, hydroxy, cyano, (C₁-C₆)alkoxy, —NR^{u}R^{v}, and (C₃-C₆)cycloalkyl;

A specific value for R^{b} is —C(═O)R^{c}.

A specific value for R^{b} is S(═O)ₙR^{d}.

A specific compound or salt is a compound of formula (Ia):

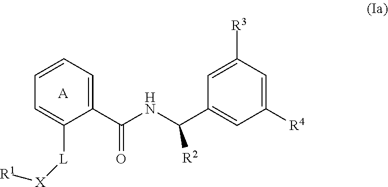

or a salt thereof.

A specific compound or salt is a compound of formula (Ib):

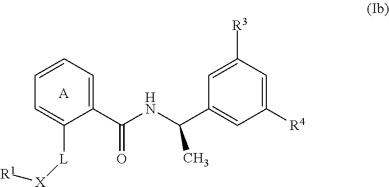

or a salt thereof.

A specific compound or salt is a compound of formula (Ic):

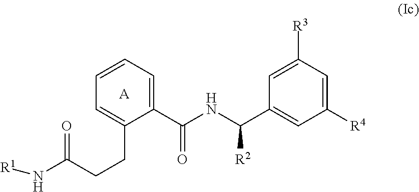

or a salt thereof.

A specific compound or salt is a compound of formula (Id):

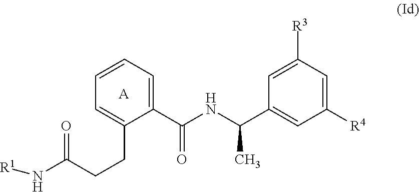

or a salt thereof.

A specific compound or salt is a compound of formula (Ie):

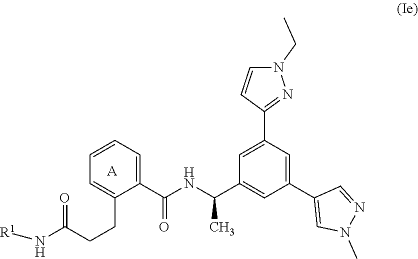

or a salt thereof.

A specific value for the group -L-X—R¹ has a structure selected from the group consisting of:

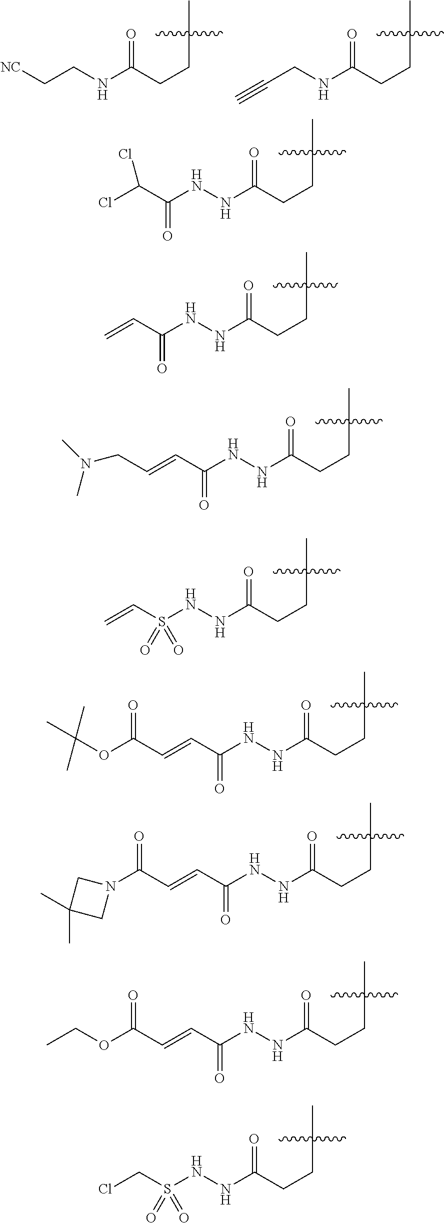

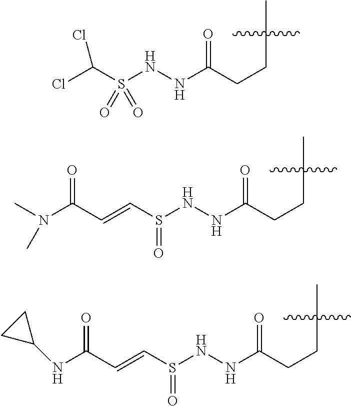 [SMILES: *CCC(=O)NNS(=O)(=O)C(Cl)Cl ; *CCC(=O)NNS(=O)/C=C/C(=O)N(C)C ; *CCC(=O)NNS(=O)/C=C/C(=O)NC1CC1]

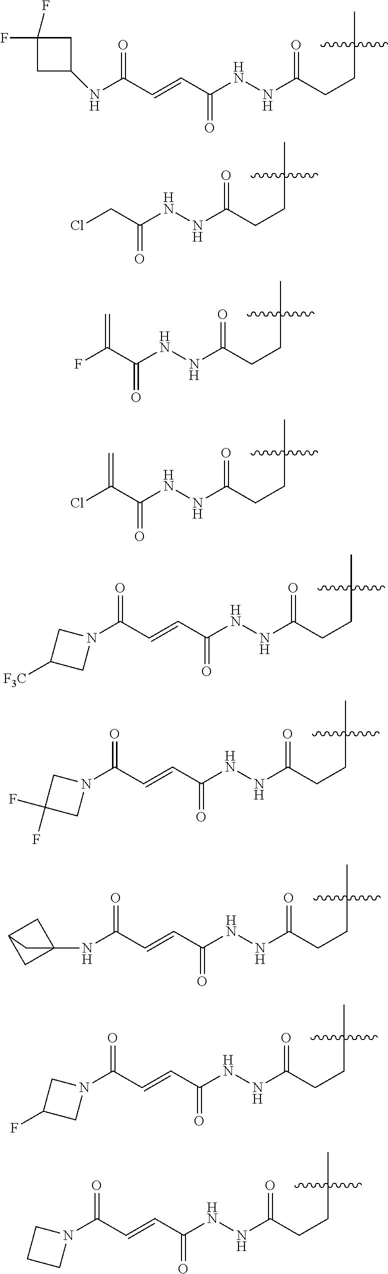

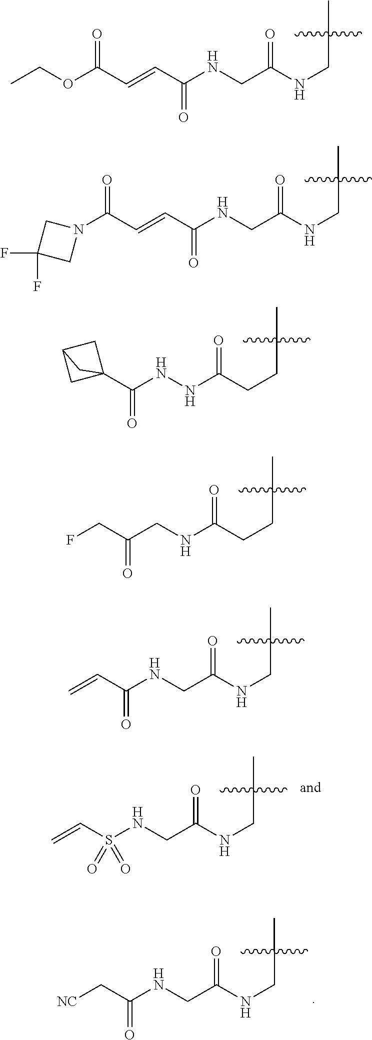

In one embodiment, the compound or salt is not:

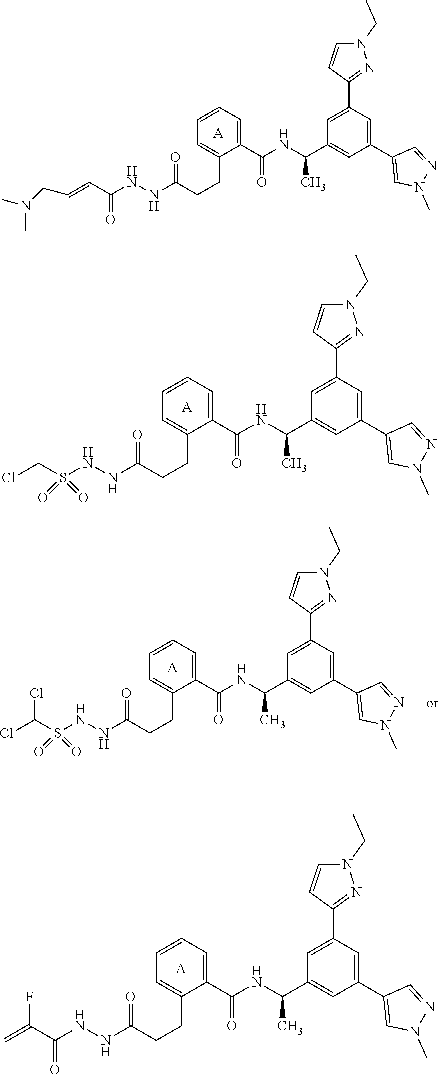

or a salt thereof.

In one embodiment, —X—R¹ is not:

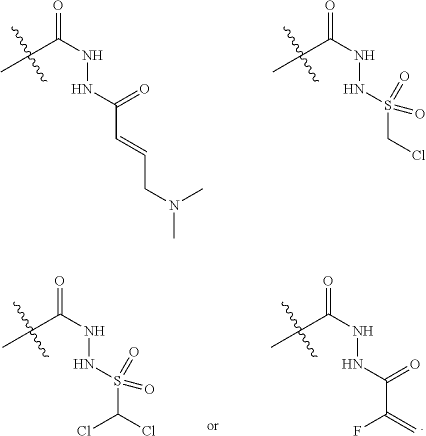

In one embodiment, R^{d} is (C₁-C₆)alkyl, (C₂-C₆)alkenyl, (C₂-C₆)alkynyl, or (C₃-C₆)cycloalkyl, wherein any (C₁-C₆)alkyl, (C₂-C₆)alkenyl, (C₂-C₆)alkynyl, and (C₃-C₆)cycloalkyl is optionally substituted with one or more groups independently selected from the group consisting of hydroxy, cyano, (C₁-C₆)alkoxy, (C₁-C₆)alkoxycarbonyl, (C₁-C₆)alkanoyloxy, oxo (═O), —NR^{y}R^{z}, carboxy, —C(═O)NR^{y}R^{z}, and (C₃-C₆)cycloalkyl;

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for the treatment of viral infections (e.g., protease inhibitors). Examples of such agents include nirmatrelvir, ensitrelvir, molnupiravir, VV116, and remdesivir. Accordingly, in one embodiment the invention also provides a composition comprising a compound of formula I, or a pharmaceutically acceptable salt thereof, at least one other therapeutic agent, and a pharmaceutically acceptable diluent or carrier. The invention also provides a kit comprising a compound of formula I, or a pharmaceutically acceptable salt thereof, at least one other therapeutic agent, packaging material, and instructions for administering the compound of formula I or the pharmaceutically acceptable salt thereof and the other therapeutic agent or agents to an animal to treat a viral infection.

The invention will now be illustrated by the following non-limiting Examples.

### EXAMPLES

### General Synthetic Procedure 1

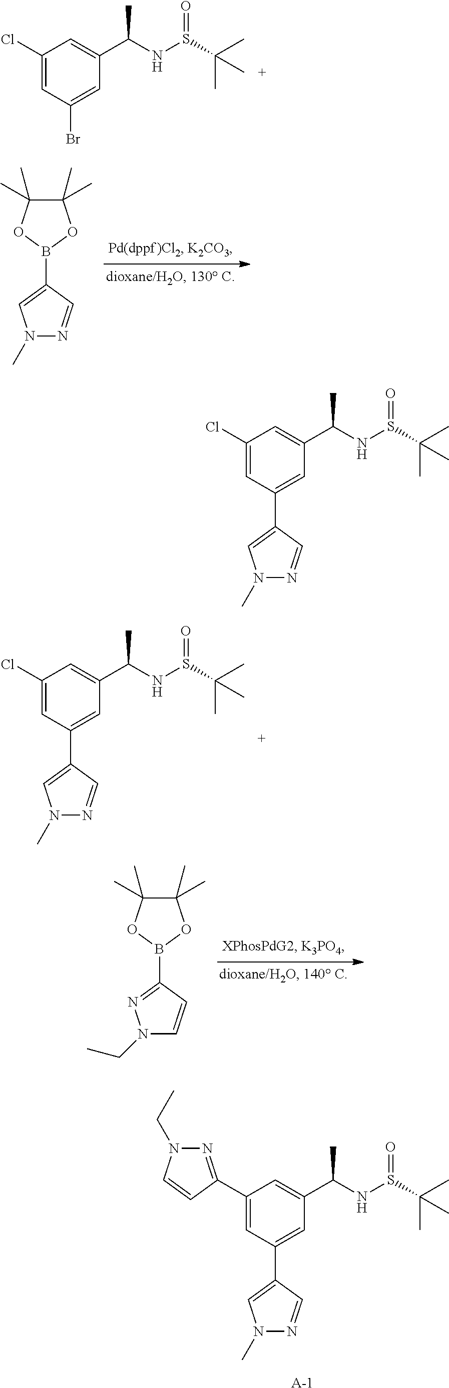

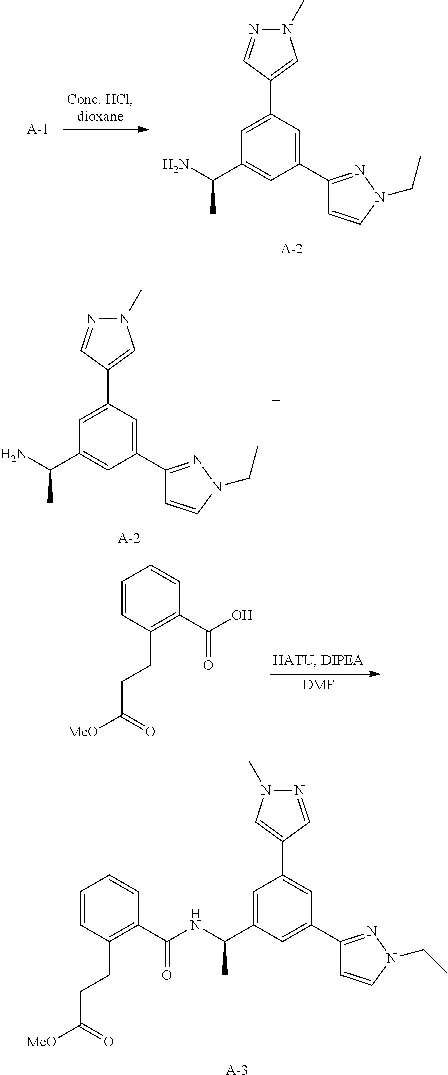

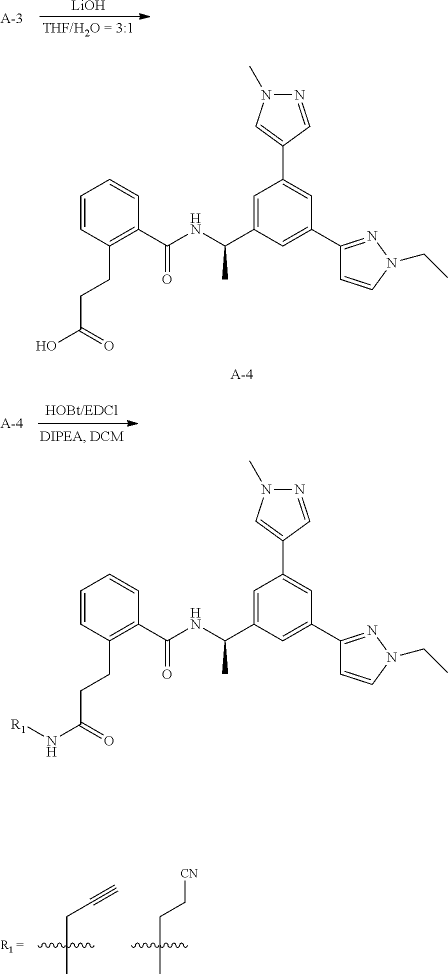

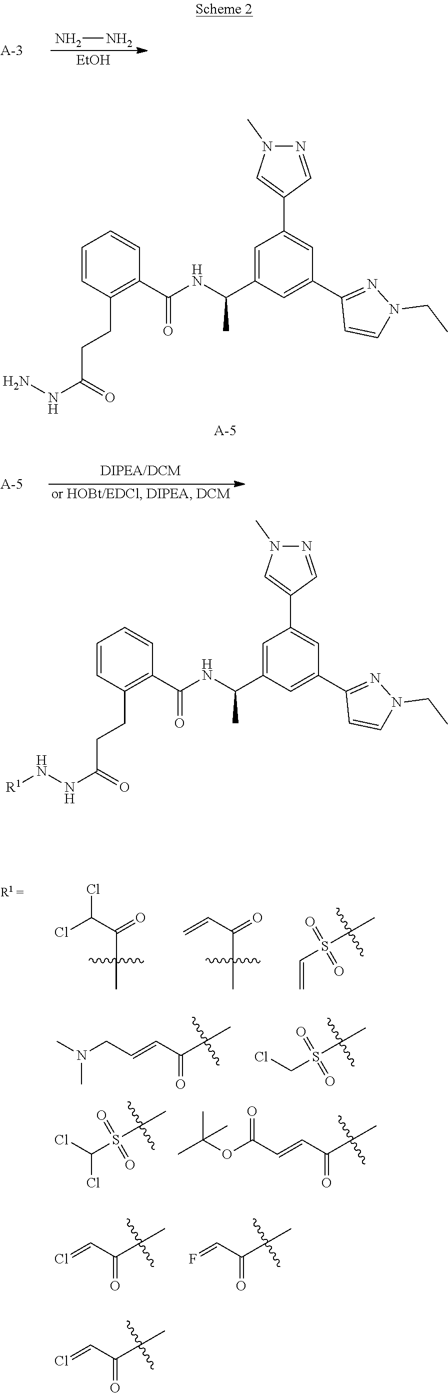

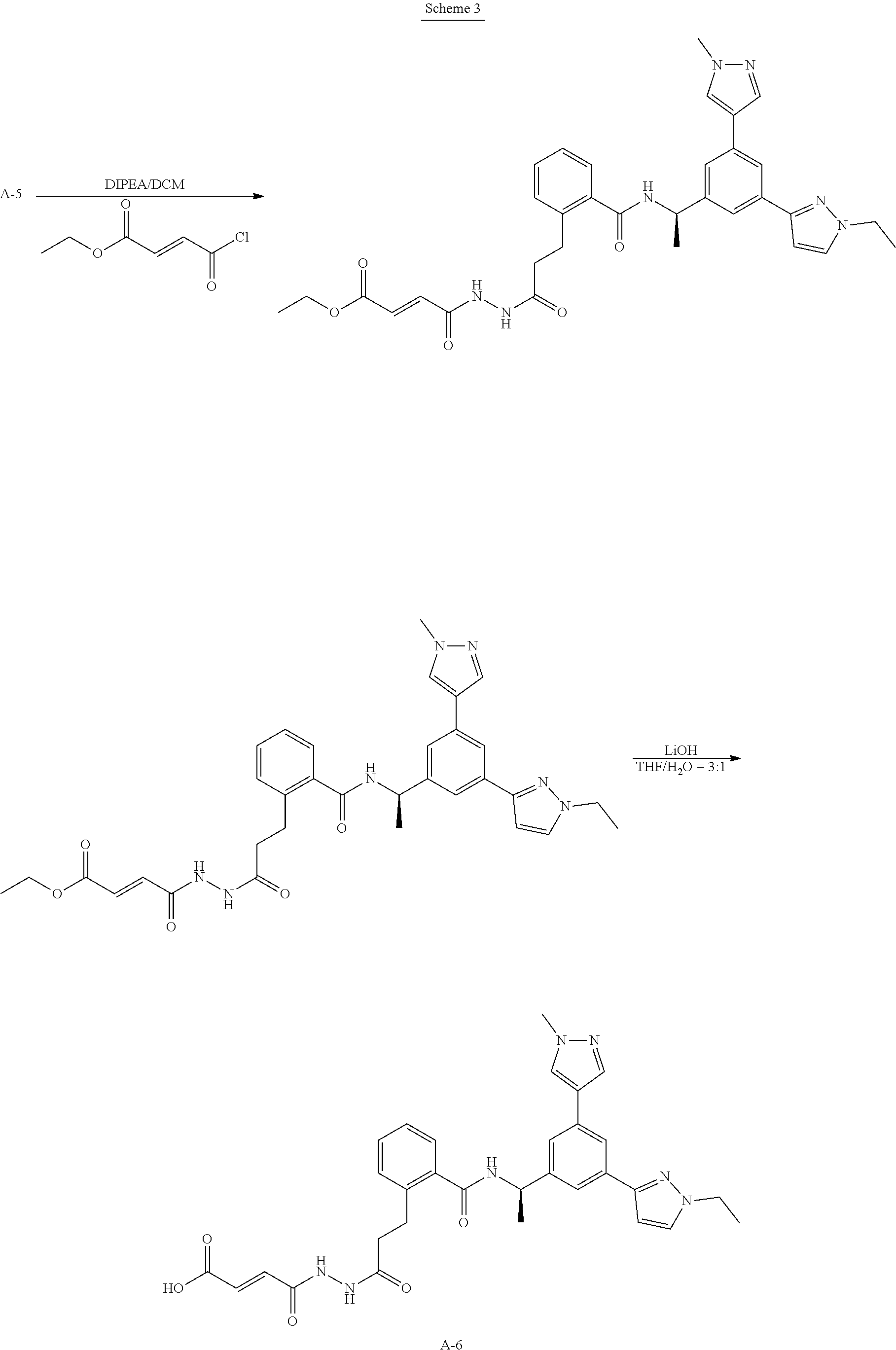

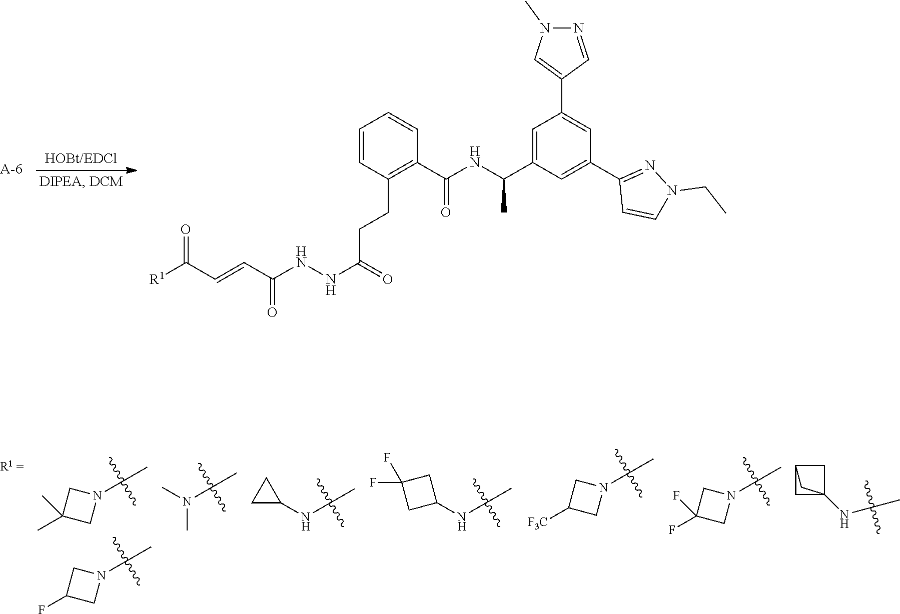

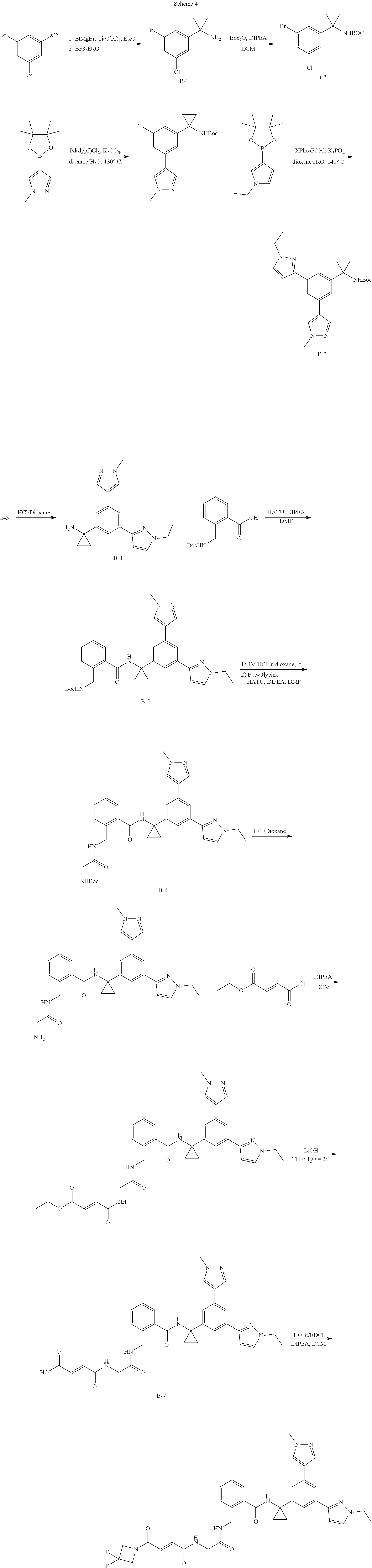

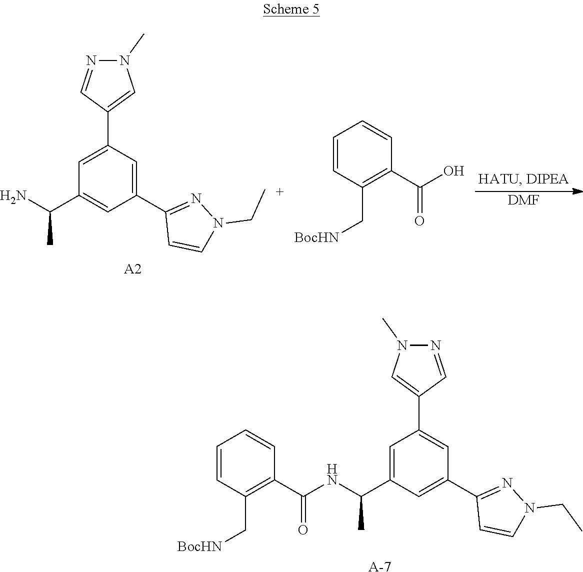

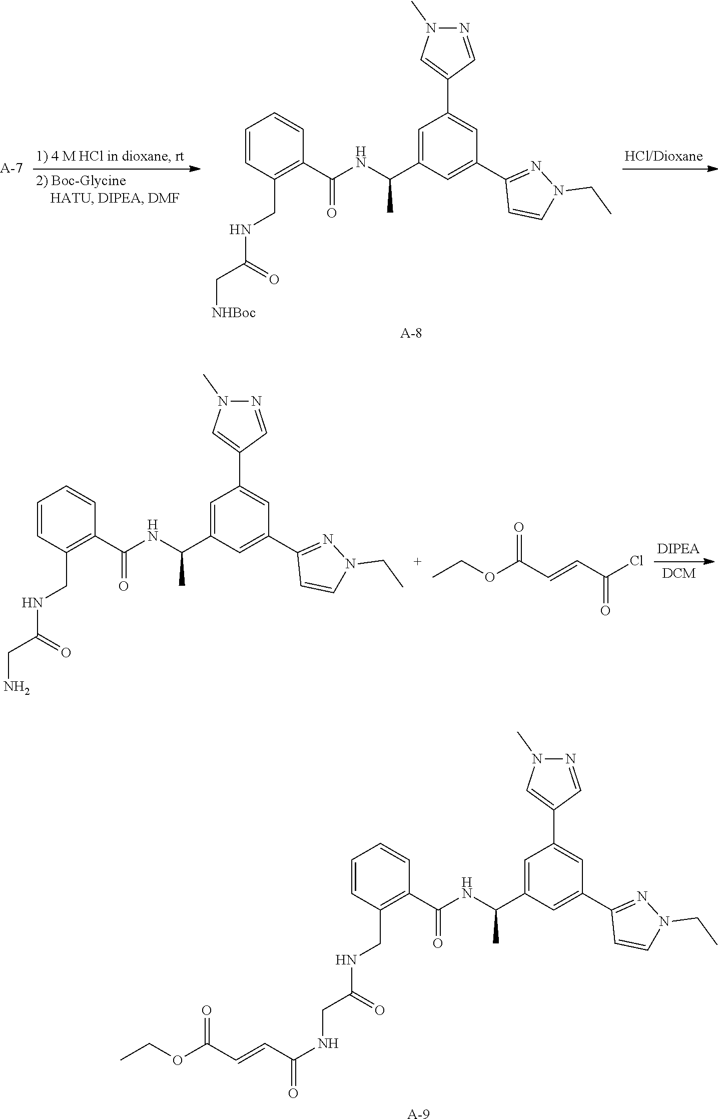

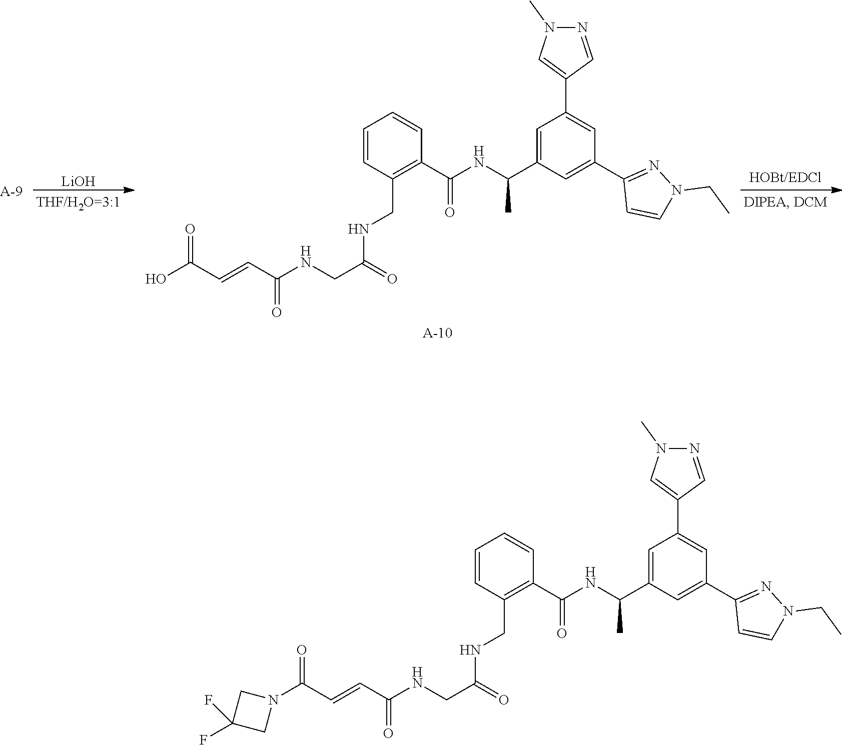 [SMILES: "A-9" ; "LiOH" ; CCn1ccc(-c2cc(-c3cnn(C)c3)cc([C@@H](C)NC(=O)c3ccccc3CNC(=O)CNC(=O)/C=C/C(=O)O)c2)n1 ; "A-10" ; CCn1ccc(-c2cc(-c3cnn(C)c3)cc([C@@H](C)NC(=O)c3ccccc3CNC(=O)CNC(=O)/C=C/C(=O)N3CC(F)(F)C3)c2)n1]

### Example 1. Synthesis of (R)-2-(3-((2-Cyanoethyl)amino)-3-oxopropyl)-N-(1-(3-(1-ethyl-1H-pyrazol-3-yl)-5-(1-methyl-1H-pyrazol-4-yl)phenyl)ethyl)benzamide

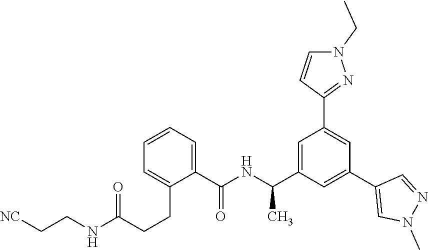 [SMILES: CCn1ccc(-c2cc(-c3cnn(C)c3)cc([C@@H](C)NC(=O)c3ccccc3CCC(=O)NCCC#N)c2)n1]

White solid, 77% yield. ¹H NMR (400 MHZ, DMSO-d₆) δ 8.81 (d, J=8.2 Hz, 1H), 8.12 (s, 1H), 7.83 (s, 1H), 7.75 (s, 1H), 7.72 (d, J=2.2 Hz, 1H), 7.63 (s, 1H), 7.45 (s, 1H), 7.28 (dd, J=7.3, 1.7 Hz, 2H), 7.22-7.16 (m, 2H), 6.68 (d, J=2.3 Hz, 1H), 5.15-5.08 (m, 1H), 4.11 (q, J=7.3 Hz, 2H), 3.82 (s, 3H), 3.04 (q, J=7.1 Hz, 2H), 2.83 (t, J=7.3 Hz, 2H), 2.72 (t, J=2.6 Hz, 1H), 2.36-2.30 (m, 2H), 2.14 (td, J=7.2, 2.7 Hz, 2H), 1.43 (d, J=7.0 Hz, 3H), 1.35 (t, J=7.3 Hz, 3H). ¹³C NMR (101 MHz, DMSO-d₆) δ 171.98, 168.72, 150.34, 146.15, 139.29, 137.66, 136.49, 134.60, 133.33, 131.14, 129.77, 129.72, 128.42, 127.71, 126.24, 122.44, 122.35, 120.85, 120.57, 102.99, 82.68, 72.41, 48.92, 46.81, 39.10, 38.21, 37.05, 28.72, 23.04, 19.17, 16.01. C₃₀H₃₃N₇O₂, HRMS calculated for m/z [M+H]⁺: 524.2774 (calculated), 527.2779 (found).

### Example 2. Synthesis of (R)-N-(1-(3-(1-Ethyl-1H-pyrazol-3-yl)-5-(1-methyl-1H-pyrazol-4-yl)phenyl)ethyl)-2-(3-oxo-3-(prop-2-yn-1-ylamino)propyl)benzamide

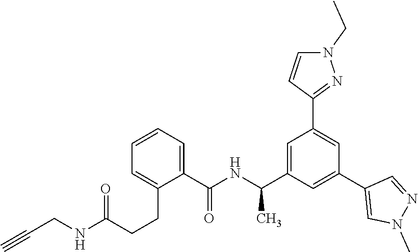 [SMILES: C#CCNC(=O)CCc1ccccc1C(=O)N[C@H](C)c1cc(-c2cnn(C)c2)cc(-c2ccn(CC)n2)c1]

White solid, 78% yield. ¹H NMR (400 MHZ, DMSO-d₆) δ 8.80 (d, J=8.2 Hz, 1H), 8.13 (d, J=6.4 Hz, 2H), 7.83 (s, 1H), 7.75 (s, 1H), 7.72 (d, J=2.3 Hz, 1H), 7.63 (s, 1H), 7.45 (s, 1H), 7.29-7.26 (m, 2H), 7.21-7.18 (m, 2H), 6.68 (d, J=2.3 Hz, 1H), 5.15-5.08 (m, 1H), 4.11 (q, J=7.3 Hz, 2H), 3.82 (s, 3H), 3.74 (dd, J=5.5, 2.5 Hz, 2H), 2.99 (t, J=2.5 Hz, 1H), 2.83 (t, J=7.8 Hz, 2H), 2.36 (dd, J=8.7, 6.9 Hz, 2H), 1.43 (d, J=7.0 Hz, 3H), 1.35 (t, J=7.3 Hz, 3H). ¹³C NMR (101 MHZ, DMSO-d₆) δ 171.64, 168.70, 150.35, 146.13, 139.25, 137.66, 136.50, 134.60, 133.33, 131.14, 129.81, 129.73, 128.43, 127.71, 126.26, 122.44, 122.35, 120.86, 120.59, 103.00, 81.68, 73.27, 48.91, 46.81, 39.10, 36.88, 28.63, 28.23, 23.02, 16.01. C₃₀H₃₂N₆O₂, HRMS calculated for m/z [M+H]⁺: 509.2265 (calculated), 509.2272 (found).

### Example 3. Synthesis of (R)-2-(3-(2-(2,2-Dichloroacetyl)hydrazineyl)-3-oxopropyl)-N-(1-(3-(1-ethyl-1H-pyrazol-3-yl)-5-(1-methyl-1H-pyrazol-4-yl)phenyl)ethyl)benzamide

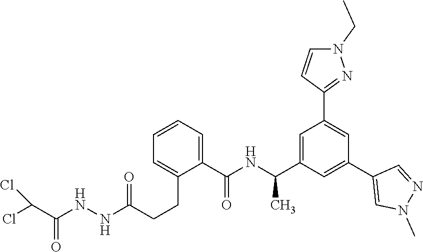 [SMILES: CCn1ccc(-c2cc(-c3cnn(C)c3)cc([C@@H](C)NC(=O)c3ccccc3CCC(=O)NNC(=O)C(Cl)Cl)c2)n1]

White solid, 85% yield. ¹H NMR (400 MHZ, DMSO-d₆) δ 10.59 (d, J=2.0 Hz, 1H), 10.16 (d, J=2.0 Hz, 1H), 8.78 (d, J=8.2 Hz, 1H), 8.12 (s, 1H), 7.84 (s, 1H), 7.75 (s, 1H), 7.72 (d, J=2.3 Hz, 1H), 7.63 (s, 1H), 7.45 (s, 1H), 7.29 (d, J=7.1 Hz, 2H), 7.27-7.20 (m, 2H), 6.68 (d, J=2.3 Hz, 1H), 6.45 (s, 1H), 5.16-5.08 (m, 1H), 4.12 (q, J=7.2 Hz, 2H), 3.81 (s, 3H), 2.87 (t, J=7.3 Hz, 2H), 2.47-2.43 (m, 2H), 1.43 (d, J=7.0 Hz, 3H), 1.35 (t, J=7.3 Hz, 3H). ¹³C NMR (101 MHZ, DMSO-d₆) δ 170.59, 168.66, 162.67, 150.34, 146.12, 138.95, 137.65, 136.51, 134.62, 133.33, 131.14, 129.91, 129.83, 128.43, 127.71, 126.41, 122.44, 122.35, 120.85, 120.60, 103.01, 65.67, 48.91, 46.81, 39.10, 35.02, 28.44, 23.00, 16.01. C₂₉H₃₁Cl₂N₇O₃, HRMS calculated for m/z [M+H]⁺: 596.1944 (calculated), 596.1951 (found).

### Example 4. Synthesis of (R)-2-(3-(2-Acryloylhydrazineyl)-3-oxopropyl)-N-(1-(3-(1-ethyl-1H-pyrazol-3-yl)-5-(1-methyl-1H-pyrazol-4-yl)phenyl)ethyl)benzamide

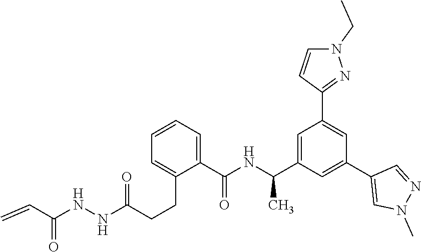 [SMILES: C=CC(=O)NNC(=O)CCc1ccccc1C(=O)N[C@H](C)c1cc(-c2cnn(C)c2)cc(-c2ccn(CC)n2)c1]

White solid, 68% yield. ¹H NMR (400 MHZ, DMSO-d₆) δ 10.01 (d, J=2.3 Hz, 1H), 9.90 (d, J=2.3 Hz, 1H), 8.79 (d, J=8.1 Hz, 1H), 8.12 (s, 1H), 7.83 (s, 1H), 7.75 (s, 1H), 7.72 (d, J=2.3 Hz, 1H), 7.63 (s, 1H), 7.45 (s, 1H), 7.27 (dd, J=12.9, 7.2 Hz, 3H), 7.21 (d, J=7.6 Hz, 1H), 6.68 (d, J=2.3 Hz, 1H), 6.21 (dd, J=17.1, 10.0 Hz, 1H), 6.11 (dd, J=17.1, 2.4 Hz, 1H), 5.62 (dd, J=10.0, 2.4 Hz, 1H), 5.15-5.08 (m, 1H), 4.11 (q, J=7.2 Hz, 2H), 3.81 (s, 3H), 2.87 (t, J=7.3 Hz, 2H), 2.47-2.43 (m, 2H), 1.43 (d, J=7.0 Hz, 3H), 1.35 (t, J=7.3 Hz, 3H). 13C NMR (101 MHZ, DMSO-d₆) δ 170.60, 168.67, 163.60, 150.34, 146.13, 139.03, 137.69, 136.51, 134.62, 133.34, 131.15, 129.87, 129.80, 129.77, 128.43, 127.69, 127.12, 126.36, 122.43, 122.36, 120.84, 120.58, 103.01, 48.91, 46.81, 39.10, 35.04, 28.51, 23.03, 16.02. C₃₀H₃₃N₇O₃, HRMS calculated for m/z [M+H]⁺: 540.2723 (calculated), 540.2731 (found).

### Example 5. Synthesis of (R,E)-2-(3-(2-(4-(Dimethylamino)but-2-enoyl)hydrazineyl)-3-oxopropyl)-N-(1-(3-(1-ethyl-1H-pyrazol-3-yl)-5-(1-methyl-1H-pyrazol-4-yl)phenyl)ethyl)benzamide

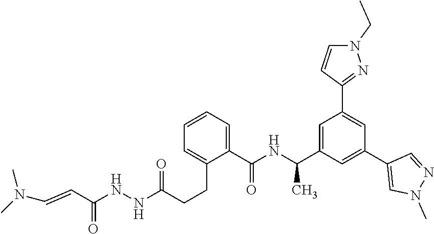 [SMILES: CCn1ccc(-c2cc(-c3cnn(C)c3)cc([C@@H](C)NC(=O)c3ccccc3CCC(=O)NNC(=O)/C=C/N(C)C)c2)n1]

White solid, 43% yield. ¹H NMR (400 MHZ, DMSO-d₆) δ 10.20 (d, J=2.2 Hz, 1H), 9.96 (d, J=2.2 Hz, 1H), 8.81 (t, J=7.4 Hz, 1H), 8.13 (s, 1H), 7.83 (s, 1H), 7.74 (d, J=9.2 Hz, 2H), 7.63 (s, 1H), 7.48-7.42 (m, 1H), 6.68 (s, 1H), 6.60 (dd, J=15.1, 7.5 Hz, 1H), 5.11 (tt, J=6.8, 3.3 Hz, 1H), 4.12 (q, J=7.2 Hz, 2H), 3.84 (d, J=7.1 Hz, 1H), 3.81 (d, J=1.4 Hz, 3H), 2.88 (t, J=8.0 Hz, 2H), 2.70 (s, 3H), 2.66 (dd, J=13.7, 3.9 Hz, 1H), 2.54-2.45 (m, 1H), 1.43 (d, J=6.9 Hz, 3H), 1.35 (t, J=7.3 Hz, 3H). ¹³C NMR (101 MHz, DMSO-d₆) δ 171.45, 170.66, 168.67, 168.64, 162.50, 159.05, 158.70, 150.33, 146.13, 139.02, 138.66, 137.66, 136.51, 134.62, 133.34, 131.99, 131.16, 129.98, 128.42, 122.42, 122.33, 120.82, 120.59, 117.93, 115.01, 103.00, 57.18, 48.92, 46.80, 42.66, 42.43, 39.11, 35.03, 34.79, 23.03, 16.01. C₃₃H₄₀N₈O₃, HRMS calculated for m/z [M+H]⁺: 597.3302 (calculated), 597.3311 (found).

### Example 6. Synthesis of (R)-N-(1-(3-(1-Ethyl-1H-pyrazol-3-yl)-5-(1-methyl-1H-pyrazol-4-yl)phenyl)ethyl)-2-(3-oxo-3-(2-(vinylsulfonyl)hydrazineyl)propyl)benzamide

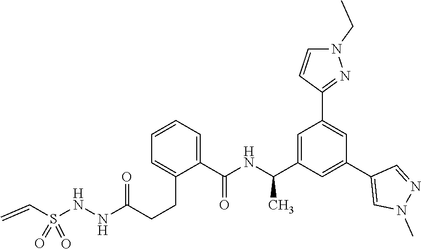 [SMILES: C=CS(=O)(=O)NNC(=O)CCc1ccccc1C(=O)N[C@H](C)c1cc(-c2cnn(C)c2)cc(-c2ccn(CC)n2)c1]

White solid, 98% yield. ¹H NMR (400 MHZ, DMSO-d₆) δ 10.03 (s, 1H), 9.47 (s, 1H), 8.77 (d, J=8.1 Hz, 1H), 8.13 (s, 1H), 7.84 (s, 1H), 7.75 (s, 1H), 7.72 (d, J=2.3 Hz, 1H), 7.63 (s, 1H), 7.45 (s, 1H), 7.28 (d, J=6.9 Hz, 2H), 7.20 (t, J=7.1 Hz, 2H), 6.68 (d, J=2.3 Hz, 1H), 6.48 (dd, J=16.5, 9.9 Hz, 1H), 5.96-5.83 (m, 1H), 5.15-5.07 (m, 1H), 4.12 (q, J=7.3 Hz, 2H), 3.82 (s, 3H), 2.91-2.77 (m, 2H), 2.36 (t, J=7.7 Hz, 2H), 1.43 (d, J=7.0 Hz, 3H), 1.35 (t, J=7.2 Hz, 3H). ¹³C NMR (101 MHz, DMSO-d₆) δ 171.40, 168.61, 150.33, 146.12, 138.79, 137.64, 136.49, 136.36, 134.61, 133.33, 131.15, 129.80, 128.43, 127.71, 127.36, 126.42, 122.43, 122.34, 120.81, 120.59, 117.02, 114.15, 103.00, 48.91, 46.81, 39.10, 34.83, 28.30, 23.03, 16.01. C₂₉H₃₃N₇O₄S, HRMS calculated for m/z [M+H]⁺: 576.2393 (calculated), 576.2400 (found).

### Example 7. Synthesis of tert-Butyl (R,E)-4-(2-(3-(2-((1-(3-(1-ethyl-1H-pyrazol-3-yl)-5-(1-methyl-1H-pyrazol-4-yl)phenyl)ethyl)carbamoyl)phenyl)propanoyl)hydrazineyl)-4-oxobut-2-enoate

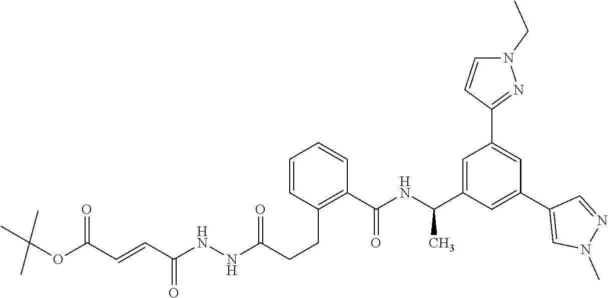 [SMILES: CCn1ccc(-c2cc(-c3cnn(C)c3)cc([C@@H](C)NC(=O)c3ccccc3CCC(=O)NNC(=O)/C=C/C(=O)OC(C)(C)C)c2)n1]

White solid, 66% yield. ¹H NMR (400 MHZ, DMSO-d₆) δ 10.45 (s, 1H), 10.11 (s, 1H), 8.79 (d, J=8.3 Hz, 1H), 8.12 (s, 1H), 7.83 (s, 1H), 7.75 (s, 1H), 7.71 (d, J=2.3 Hz, 1H), 7.63 (s, 1H), 7.45 (s, 1H), 7.29 (d, J=7.2 Hz, 2H), 7.26-7.19 (m, 2H), 6.90 (d, J=15.5 Hz, 1H), 6.68 (d, J=2.3 Hz, 1H), 6.50 (d, J=15.5 Hz, 1H), 5.12 (p, J=7.1 Hz, 1H), 4.11 (q, J=7.3 Hz, 2H), 3.81 (s, 3H), 2.88 (t, J=7.4 Hz, 2H), 2.46 (d, J=8.0 Hz, 2H), 1.43 (d, J=7.0 Hz, 3H), 1.39 (s, 9H), 1.35 (t, J=7.3 Hz, 3H). ¹³C NMR (101 MHz, DMSO-d₆) δ 170.33, 168.64, 164.44, 161.73, 150.33, 146.10, 138.99, 137.64, 136.50, 134.66, 134.60, 133.33, 131.78, 131.12, 129.87, 129.79, 128.40, 127.70, 126.37, 122.42, 122.34, 120.83, 120.58, 102.99, 81.61, 48.91, 46.79, 39.08, 35.01, 28.49, 28.06, 23.01, 16.00. C₃₅H₄₁N₇O₅, HRMS calculated for m/z [M+H]⁺: 640.3247 (calculated), 640.3261 (found).

### Example 8. Synthesis of (R,E)-2-(3-(2-(4-(3,3-Dimethylazetidin-1-yl)-4-oxobut-2-enoyl)hydrazineyl)-3-oxopropyl)-N-(1-(3-(1-ethyl-1H-pyrazol-3-yl)-5-(1-methyl-1H-pyrazol-4-yl)phenyl)ethyl)benzamide

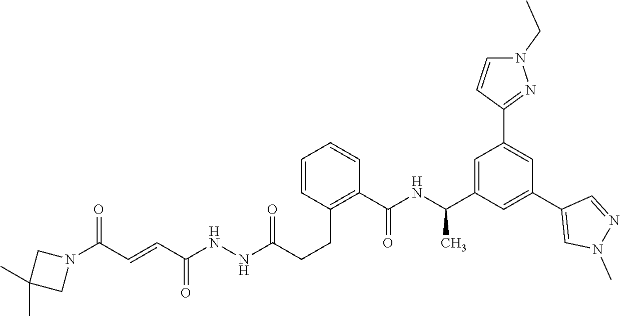 [SMILES: CCn1ccc(-c2cc(-c3cnn(C)c3)cc([C@@H](C)NC(=O)c3ccccc3CCC(=O)NNC(=O)/C=C/C(=O)N3CC(C)(C)C3)c2)n1]

White solid, 68% yield. ¹H NMR (400 MHZ, DMSO-d₆) δ 10.31 (s, 1H), 10.02 (s, 1H), 8.78 (d, J=8.1 Hz, 1H), 8.12 (s, 1H), 7.83 (s, 1H), 7.75 (s, 1H), 7.71 (d, J=2.2 Hz, 1H), 7.63 (s, 1H), 7.45 (s, 1H), 7.28 (p, J=6.9 Hz, 3H), 7.20 (t, J=7.3 Hz, 1H), 6.79 (s, 1H), 6.68 (d, J=2.3 Hz, 1H), 5.16-5.08 (m, 1H), 4.11 (q, J=7.3 Hz, 2H), 3.87 (s, 2H), 3.81 (s, 3H), 3.55 (s, 2H), 2.87 (q, J=7.1 Hz, 2H), 2.46 (d, J=7.8 Hz, 2H), 1.43 (d, J=7.0 Hz, 3H), 1.35 (t, J=7.3 Hz, 3H), 1.15 (s, 6H). ¹³C NMR (101 MHz, DMSO-d₆) δ 170.50, 168.67, 163.97, 162.56, 150.34, 146.13, 139.01, 137.67, 136.51, 134.62, 133.34, 131.29, 131.14, 129.88, 129.81, 129.24, 128.42, 127.71, 126.37, 122.43, 122.35, 120.84, 120.59, 103.01, 62.21, 60.21, 48.92, 46.81, 39.10, 35.03, 30.99, 28.49, 26.97, 23.03, 16.02. C₃₆H₄₂N₈O₄, HRMS calculated for m/z [M+H]⁺: 651.3407 (calculated), 651.3411 (found).

### Example 9. Synthesis of Ethyl (R,E)-4-(2-(3-(2-((1-(3-(1-ethyl-1H-pyrazol-3-yl)-5-(1-methyl-1H-pyrazol-4-yl)phenyl)ethyl)carbamoyl)phenyl)propanoyl)hydrazineyl)-4-oxobut-2-enoate

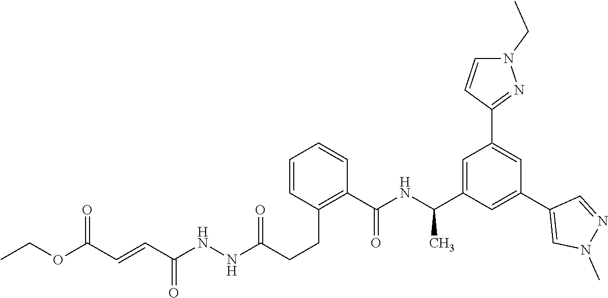 [SMILES: CCOC(=O)/C=C/C(=O)NNC(=O)CCc1ccccc1C(=O)N[C@H](C)c1cc(-c2cnn(C)c2)cc(-c2ccn(CC)n2)c1]

White solid, 68% yield. ¹H NMR (400 MHZ, DMSO-d₆) δ 10.47 (d, J=2.5 Hz, 1H), 10.12 (d, J=2.3 Hz, 1H), 8.78 (d, J=8.2 Hz, 1H), 8.12 (s, 1H), 7.83 (s, 1H), 7.75 (s, 1H), 7.71 (d, J=2.3 Hz, 1H), 7.63 (s, 1H), 7.45 (d, J=1.7 Hz, 1H), 7.28 (p, J=6.7 Hz, 3H), 7.20 (t, J=7.3 Hz, 1H), 6.99 (d, J=15.5 Hz, 1H), 6.68 (d, J=2.3 Hz, 1H), 6.59 (d, J=15.5 Hz, 1H), 5.16-5.08 (m, 1H), 4.16-4.08 (m, 4H), 3.81 (s, 3H), 2.87 (d, J=7.8 Hz, 2H), 2.46 (t, J=7.7 Hz, 2H), 1.43 (d, J=7.0 Hz, 3H), 1.35 (t, J=7.3 Hz, 3H), 1.17 (t, J=7.1 Hz, 3H). ¹³C NMR (101 MHZ, DMSO-d₆) δ 170.37, 168.66, 165.21, 161.61, 150.35, 146.11, 139.00, 137.66, 136.52, 135.39, 134.63, 133.35, 131.13, 130.11, 129.89, 129.81, 128.41, 127.71, 126.38, 122.44, 122.35, 120.85, 120.60, 103.00, 61.28, 48.92, 46.81, 39.10, 35.02, 28.50, 23.02, 16.01, 14.45. C₃₃H₃₇N₇O₅, HRMS calculated for m/z [M+H]⁺: 612.2934 (calculated), 612.2942 (found).

### Example 10. Synthesis of (R)-2-(3-(2-((Chloromethyl)sulfonyl)hydrazineyl)-3-oxopropyl)-N-(1-(3-(1-ethyl-1H-pyrazol-3-yl)-5-(1-methyl-1H-pyrazol-4-yl)phenyl)ethyl)benzamide

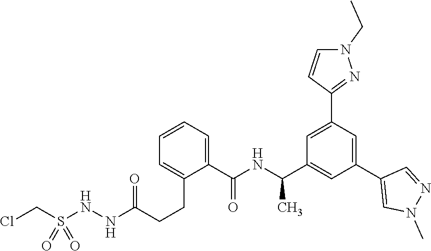 [SMILES: CCn1ccc(-c2cc(-c3cnn(C)c3)cc([C@@H](C)NC(=O)c3ccccc3CCC(=O)NNS(=O)(=O)CCl)c2)n1]

White solid, 86% yield. ¹H NMR (400 MHZ, DMSO-d₆) δ 10.09 (s, 1H), 9.91 (s, 1H), 8.78 (d, J=8.2 Hz, 1H), 8.12 (s, 1H), 7.84 (s, 1H), 7.75 (s, 1H), 7.72 (d, J=2.3 Hz, 1H), 7.63 (s, 1H), 7.45 (s, 1H), 7.29 (t, J=6.8 Hz, 2H), 7.22 (d, J=7.6 Hz, 2H), 6.68 (d, J=2.3 Hz, 1H), 5.15-5.08 (m, 1H), 4.70 (s, 2H), 4.12 (q, J=7.3 Hz, 2H), 3.82 (s, 3H), 2.87 (t, J=7.7 Hz, 2H), 2.42 (d, J=8.3 Hz, 2H), 1.43 (d, J=7.0 Hz, 3H). ¹³C NMR (101 MHz, DMSO-d₆) δ 172.16, 168.62, 150.34, 146.13, 138.86, 137.61, 136.50, 134.61, 133.33, 131.14, 129.87, 129.81, 128.43, 127.78, 126.46, 122.43, 122.35, 120.82, 120.60, 103.00, 56.23, 48.93, 46.81, 39.11, 34.86, 28.34, 23.01, 16.01. C₂₈H₃₂ClN₇O₄S, HRMS calculated for m/z [M+H]⁺: 598.2003 (calculated), 598.2010 (found).

### Example 11. Synthesis of (R)-2-(3-(2-((dichloromethyl)sulfonyl)hydrazineyl)-3-oxopropyl)-N-(1-(3-(1-Ethyl-1H-pyrazol-3-yl)-5-(1-methyl-1H-pyrazol-4-yl)phenyl)ethyl)benzamide

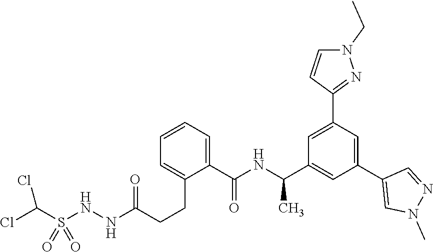 [SMILES: CCn1ccc(-c2cc(-c3cnn(C)c3)cc([C@@H](C)NC(=O)c3ccccc3CCC(=O)NNS(=O)(=O)C(Cl)Cl)c2)n1]

White solid, 68% yield. ¹H NMR (400 MHZ, DMSO-d₆) δ 10.44 (s, 1H), 10.12 (s, 1H), 8.79 (d, J=8.1 Hz, 1H), 8.12 (s, 1H), 7.84 (s, 1H), 7.75 (s, 1H), 7.72 (s, 1H), 7.63 (s, 1H), 7.45 (s, 1H), 7.29 (t, J=6.8 Hz, 2H), 7.22 (d, J=8.4 Hz, 2H), 6.93 (s, 1H), 6.68 (s, 1H), 5.15-5.08 (m, 1H), 4.12 (q, J=7.5 Hz, 2H), 3.82 (s, 3H), 2.87 (t, J=7.7 Hz, 2H), 2.45-2.41 (m, 2H), 1.43 (d, J=7.1 Hz, 3H), 1.35 (t, J=7.3 Hz, 3H). ¹³C NMR (101 MHZ, DMSO-d₆) δ 172.42, 168.63, 150.33, 146.13, 138.86, 137.68, 137.58, 136.50, 134.61, 133.33, 131.15, 129.91, 129.85, 128.44, 127.77, 126.46, 122.43, 122.35, 120.83, 120.60, 103.01, 78.80, 48.92, 46.81, 39.11, 34.86, 28.36, 23.00, 16.01. C₂₈H₃₁Cl₂N₇O₄S, HRMS calculated for m/z [M+H]⁺: 632.1614 (calculated), 632.1621 (found).

### Example 12. Synthesis of (R,E)-2-(3-(2-(4-(Dimethylamino)-4-oxobut-2-enoyl)hydrazineyl)-3-oxopropyl)-N-(1-(3-(1-ethyl-1H-pyrazol-3-yl)-5-(1-methyl-1H-pyrazol-4-yl)phenyl)ethyl)benzamide

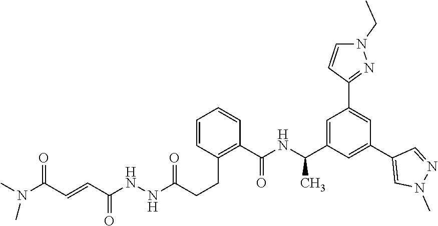 [SMILES: CCn1ccc(-c2cc(-c3cnn(C)c3)cc([C@@H](C)NC(=O)c3ccccc3CCC(=O)NNC(=O)/C=C/C(=O)N(C)C)c2)n1]

White solid, 67% yield. ¹H NMR (400 MHZ, DMSO-d₆) δ 10.29 (s, 1H), 9.99 (s, 1H), 8.78 (d, J=8.2 Hz, 1H), 8.12 (s, 1H), 7.84 (s, 1H), 7.75 (s, 1H), 7.71 (d, J=2.3 Hz, 1H), 7.64 (s, 1H), 7.45 (s, 1H), 7.33-7.26 (m, 3H), 7.25-7.17 (m, 2H), 6.77 (d, J=15.2 Hz, 1H), 6.68 (d, J=2.2 Hz, 1H), 5.16-5.09 (m, 1H), 4.11 (q, J=7.3 Hz, 2H), 3.81 (s, 3H), 2.87 (d, J=7.7 Hz, 2H), 2.84 (s, 3H), 2.47 (d, J=7.8 Hz, 2H), 1.43 (d, J=7.0 Hz, 3H), 1.35 (t, J=7.2 Hz, 3H). ¹³C NMR (101 MHZ, DMSO-d₆) δ 170.57, 168.68, 164.64, 162.81, 150.34, 146.13, 139.00, 137.66, 136.50, 134.61, 133.33, 132.01, 131.13, 129.87, 129.81, 128.43, 127.70, 126.37, 122.44, 122.36, 120.86, 120.59, 103.01, 48.92, 46.81, 39.09, 37.41, 35.67, 35.03, 28.49, 23.01, 16.00. C₃₃H₃₈N₈O₄, HRMS calculated for m/z [M+H]⁺: 611.3094 (calculated), 611.3102 (found).

### Example 13. Synthesis of (R,E)-2-(3-(2-(4-(Cyclopropylamino)-4-oxobut-2-enoyl)hydrazineyl)-3-oxopropyl)-N-(1-(3-(1-ethyl-1H-pyrazol-3-yl)-5-(1-methyl-1H-pyrazol-4-yl)phenyl)ethyl)benzamide

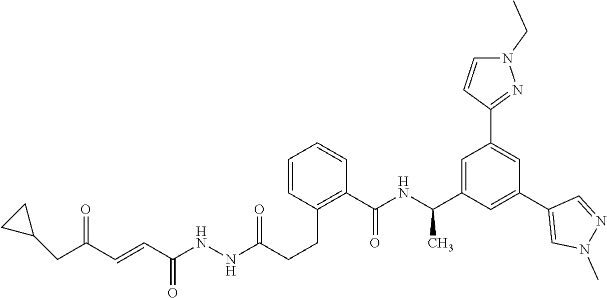 [SMILES: CCn1ccc(-c2cc(-c3cnn(C)c3)cc([C@@H](C)NC(=O)c3ccccc3CCC(=O)NNC(=O)/C=C/C(=O)CC3CC3)c2)n1]

White solid, 44% yield. ¹H NMR (400 MHZ, DMSO-d₆) δ 10.27 (s, 1H), 9.97 (s, 1H), 8.78 (d, J=8.1 Hz, 1H), 8.43 (d, J=4.7 Hz, 1H), 8.12 (s, 1H), 7.83 (s, 1H), 7.74 (s, 1H), 7.71 (d, J=2.3 Hz, 1H), 7.63 (s, 1H), 7.45 (s, 1H), 7.28 (p, J=7.4 Hz, 3H), 7.20 (t, J=7.3 Hz, 1H), 6.77 (d, J=2.6 Hz, 1H), 6.68 (d, J=2.3 Hz, 1H), 5.15-5.08 (m, 1H), 4.11 (q, J=7.3 Hz, 2H), 3.81 (s, 3H), 2.87 (t, J=6.6 Hz, 2H), 2.69 (td, J=7.4, 3.7 Hz, 1H), 1.43 (d, J=7.0 Hz, 3H), 1.35 (t, J=7.3 Hz, 3H), 0.64-0.59 (m, 1H), 0.38 (q, J=4.6 Hz, 1H). ¹³C NMR (101 MHZ, DMSO-d₆) δ 170.49, 168.66, 164.87, 162.76, 150.34, 146.12, 139.01, 137.66, 136.51, 134.61, 134.26, 133.34, 131.14, 130.20, 129.88, 129.81, 128.42, 127.70, 126.37, 122.43, 122.35, 120.84, 120.59, 103.01, 48.91, 46.81, 39.10, 35.03, 28.49, 23.04, 23.01, 16.01, 6.30, 6.04. C₃₄H₃₈N₈O₄, HRMS calculated for m/z [M+H]⁺: 623.3094 (calculated), 611.3100 (found).

Example 14. Synthesis of (R,E)-2-(3-(2-(4-((3,3-Difluorocyclobutyl)amino)-4-oxobut-2-enoyl)hydrazineyl)-3-oxopropyl)-N-(1-(3-(1-ethyl-1H-pyrazol-3-yl)-5-(1-methyl-1H-pyrazol-4-yl)phenyl)ethyl)benzamide

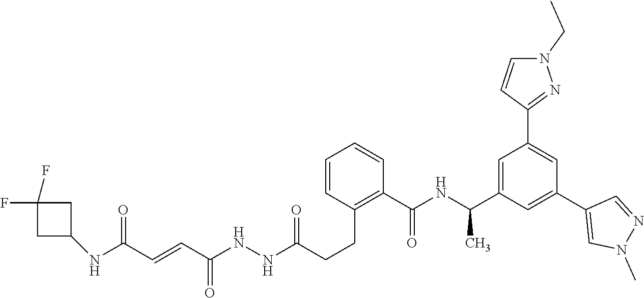 [SMILES: CCn1ccc(-c2cc(-c3cnn(C)c3)cc([C@@H](C)NC(=O)c3ccccc3CCC(=O)NNC(=O)/C=C/C(=O)NC3CC(F)(F)C3)c2)n1]

White solid, 79% yield. ¹H NMR (400 MHZ, DMSO-d₆) δ 10.32 (s, 1H), 10.00 (s, 1H), 8.87 (d, J=6.5 Hz, 1H), 8.78 (d, J=8.2 Hz, 1H), 8.12 (s, 1H), 7.83 (s, 1H), 7.74 (s, 1H), 7.72 (d, J=2.3 Hz, 1H), 7.63 (s, 1H), 7.45 (s, 1H), 7.33-7.18 (m, 5H), 6.80 (s, 2H), 6.68 (d, J=2.3 Hz, 1H), 5.15-5.08 (m, 1H), 4.12 (q, J=7.2 Hz, 2H), 3.81 (s, 3H), 2.92-2.84 (m, 4H), 2.49 (d, J=19.5 Hz, 2H), 1.43 (d, J=7.0 Hz, 3H), 1.35 (t, J=7.3 Hz, 3H). ¹³C NMR (101 MHZ, DMSO-d₆) δ 170.49, 168.67, 163.79, 162.57, 150.34, 146.11, 139.01, 137.65, 136.51, 134.62, 133.78, 133.34, 131.13, 130.95, 129.88, 129.81, 128.41, 127.70, 126.37, 122.44, 122.36, 120.86, 120.60, 103.01, 48.91, 46.81, 42.69, 42.46, 42.25, 39.09, 35.04, 28.49, 23.00, 16.00. C₃₅H₃₈F₂N₈O₄, HRMS calculated for m/z [M+H]⁺: 673.3062 (calculated), 673.3071 (found).

### Example 15 Synthesis of (R)-2-(3-(2-(2-Chloroacetyl)hydrazineyl)-3-oxopropyl)-N-(1-(3-(1-ethyl-1H-pyrazol-3-yl)-5-(1-methyl-1H-pyrazol-4-yl)phenyl)ethyl)benzamide

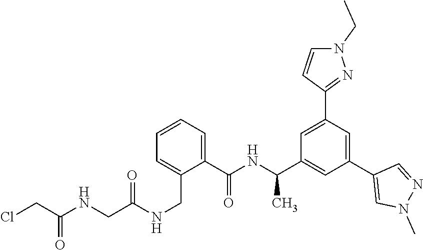 [SMILES: CCn1ccc(-c2cc(-c3cnn(C)c3)cc([C@@H](C)NC(=O)c3ccccc3CNC(=O)CNC(=O)CCl)c2)n1]

White solid, 88% yield. ¹H NMR (400 MHZ, DMSO-d₆) δ 10.14 (s, 1H), 9.92 (s, 1H), 8.78 (d, J=8.2 Hz, 1H), 8.12 (s, 1H), 7.83 (s, 1H), 7.75 (s, 1H), 7.72 (d, J=2.3 Hz, 1H), 7.63 (s, 1H), 7.45 (s, 1H), 7.32-7.18 (m, 5H), 6.68 (d, J=2.3 Hz, 1H), 5.15-5.08 (m, 1H), 4.11 (q, J=7.3 Hz, 2H), 4.05 (s, 2H), 3.81 (s, 3H), 2.88 (q, J=7.1 Hz, 2H), 1.43 (d, J=7.0 Hz, 3H), 1.35 (t, J=7.3 Hz, 3H). ¹³C NMR (101 MHz, DMSO-d₆) δ 170.66, 168.68, 165.16, 150.34, 146.13, 138.99, 137.65, 136.51, 134.61, 133.33, 131.15, 129.89, 129.81, 128.43, 127.69, 126.37, 122.43, 122.35, 120.85, 120.59, 103.01, 48.91, 46.81, 41.34, 39.10, 35.00, 28.46, 23.01, 16.01. C₂₉H₃₂ClN₇O₃, HRMS calculated for m/z [M+H]⁺: 562.2333 (calculated), 562.2340 (found).

### Example 16 Synthesis of (R)-N-(1-(3-(1-Ethyl-1H-pyrazol-3-yl)-5-(1-methyl-1H-pyrazol-4-yl)phenyl)ethyl)-2-(3-(2-(2-fluoroacryloyl)hydrazineyl)-3-oxopropyl)benzamide

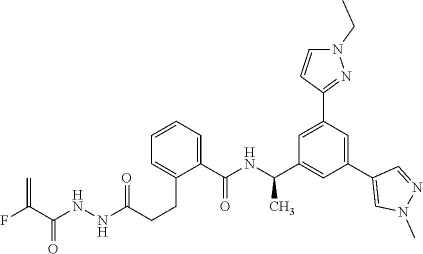 [SMILES: C=C(F)C(=O)NNC(=O)CCc1ccccc1C(=O)N[C@H](C)c1cc(-c2cnn(C)c2)cc(-c2ccn(CC)n2)c1]

White solid, 63% yield. ¹H NMR (400 MHZ, DMSO-d₆) δ 10.28 (s, 1H), 9.83 (s, 1H), 8.78 (d, J=8.1 Hz, 1H), 8.12 (s, 1H), 7.83 (s, 1H), 7.75 (s, 1H), 7.72 (s, 1H), 7.63 (s, 1H), 7.45 (s, 1H), 7.29 (d, J=7.7 Hz, 3H), 7.20 (t, J=7.2 Hz, 2H), 6.68 (s, 1H), 5.53 (dd, J=47.9, 3.6 Hz, 2H), 5.29 (dd, J=16.1, 3.7 Hz, 2H), 5.15-5.08 (m, 1H), 4.11 (q, J=7.3 Hz, 2H), 3.81 (s, 3H), 2.88 (td, J=7.0, 2.8 Hz, 2H), 2.45 (t, J=6.5 Hz, 2H), 1.43 (d, J=7.0 Hz, 3H), 1.35 (t, J=7.3 Hz, 3H). ¹³C NMR (101 MHz, DMSO-d₆) δ 171.10, 168.68, 150.34, 146.14, 139.02, 137.66, 136.51, 134.61, 133.34, 131.15, 129.92, 129.84, 128.43, 127.68, 126.37, 122.43, 122.36, 120.84, 120.58, 103.01, 48.91, 46.81, 39.16, 39.09, 35.13, 28.48, 26.01, 23.01, 21.49, 16.01. C₃₀H₃₂FN₇O₃, HRMS calculated for m/z [M+H]⁺: 558.2629 (calculated), 558.2637 (found).

### Example 17 Synthesis of (R)-2-(3-(2-(2-Chloroacryloyl)hydrazineyl)-3-oxopropyl)-N-(1-(3-(1-ethyl-1H-pyrazol-3-yl)-5-(1-methyl-1H-pyrazol-4-yl)phenyl)ethyl)

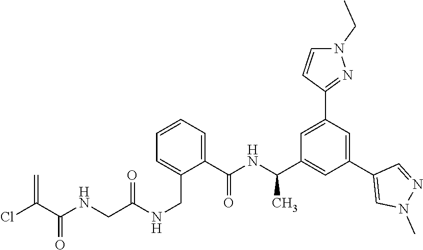 [SMILES: C=C(Cl)C(=O)NCC(=O)NCc1ccccc1C(=O)N[C@H](C)c1cc(-c2cnn(C)c2)cc(-c2ccn(CC)n2)c1]

White solid, 59% yield. ¹H NMR (400 MHZ, DMSO-d₆) δ 10.18 (s, 1H), 9.86 (s, 1H), 8.78 (d, J=8.6 Hz, 1H), 8.12 (s, 1H), 7.83 (s, 1H), 7.75 (s, 1H), 7.72 (s, 1H), 7.64 (s, 1H), 7.45 (s, 1H), 7.34-7.27 (m, 2H), 7.22 (q, J=9.7, 7.3 Hz, 1H), 6.68 (s, 1H), 6.28 (s, 1H), 5.95 (s, 1H), 5.15-5.08 (m, 1H), 4.12 (q, J=7.3 Hz, 2H), 3.81 (s, 3H), 2.89 (q, J=9.9, 8.8 Hz, 2H), 1.43 (d, J=6.9 Hz, 3H), 1.35 (t, J=7.2 Hz, 3H). ¹³C NMR (101 MHZ, DMSO-d₆) δ 171.08, 168.65, 160.88, 150.31, 146.12, 139.00, 137.64, 136.48, 134.58, 133.31, 132.23, 131.12, 129.89, 128.41, 127.67, 126.35, 123.08, 122.40, 122.33, 120.82, 120.57, 102.98, 48.88, 46.79, 39.07, 35.11, 28.46, 22.98, 15.99. C₃₀H₃₂ClN₇O₃, 574.2333 (calculated), 574.2342 (found).

Example 18 Synthesis of (R,E)-N-(1-(3-(1-Ethyl-1H-pyrazol-3-yl)-5-(1-methyl-1H-pyrazol-4-yl)phenyl)ethyl)-2-(3-oxo-3-(2-(4-oxo-4-(3-(trifluoromethyl)azetidin-1-yl)but-2-enoyl)hydrazineyl)propyl)benzamide

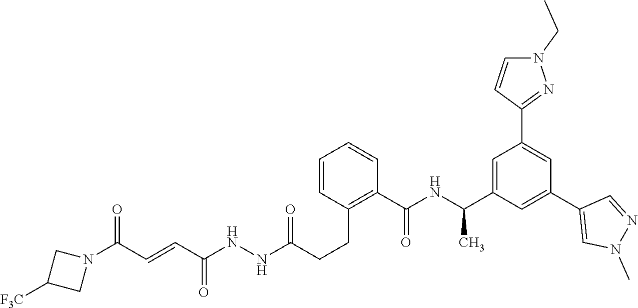 [SMILES: CCn1ccc(-c2cc(-c3cnn(C)c3)cc([C@@H](C)NC(=O)c3ccccc3CCC(=O)NNC(=O)/C=C/C(=O)N3CC(C(F)(F)F)C3)c2)n1]

White solid, 70% yield. ¹H NMR (400 MHZ, DMSO-d₆) δ 10.35 (s, 1H), 10.05 (s, 1H), 8.79 (d, J=8.2 Hz, 1H), 8.12 (s, 1H), 7.84 (s, 1H), 7.75 (s, 1H), 7.71 (d, J=2.3 Hz, 1H), 7.64 (s, 1H), 7.45 (s, 1H), 7.32-7.18 (m, 4H), 6.83 (s, 1H), 6.82 (s, 1H), 6.68 (d, J=2.3 Hz, 1H), 5.16-5.09 (m, 1H), 4.47 (t, J=9.2 Hz, 1H), 4.28 (dd, J=9.6, 5.4 Hz, 1H), 4.16-4.08 (m, 3H), 3.87 (dd, J=10.9, 5.4 Hz, 1H), 3.81 (s, 3H), 2.88 (t, J=7.3 Hz, 2H), 2.49-2.44 (m, 2H), 1.43 (d, J=7.0 Hz, 3H), 1.35 (t, J=7.3 Hz, 3H). ¹³C NMR (101 MHZ, DMSO-d₆) δ 170.46, 168.68, 164.00, 162.29, 150.34, 146.13, 139.00, 137.66, 136.50, 134.61, 133.34, 132.13, 131.14, 129.87, 129.81, 128.52, 128.43, 127.71, 126.37, 122.43, 122.36, 120.85, 120.59, 103.01, 49.84, 48.92, 47.78, 46.81, 39.08, 35.02, 28.48, 23.02, 16.00. C₃₅H₃₇F₃N₈O₄, HRMS calculated for m/z [M+H]⁺: 691.2968 (calculated), 691.2977 (found).

### Example 19 Synthesis of (R,E)-2-(3-(2-(4-(3,3-Difluoroazetidin-1-yl)-4-oxobut-2-enoyl)hydrazineyl)-3-oxopropyl)-N-(1-(3-(1-ethyl-1H-pyrazol-3-yl)-5-(1-methyl-1H-pyrazol-4-yl)phenyl)ethyl)benzamide

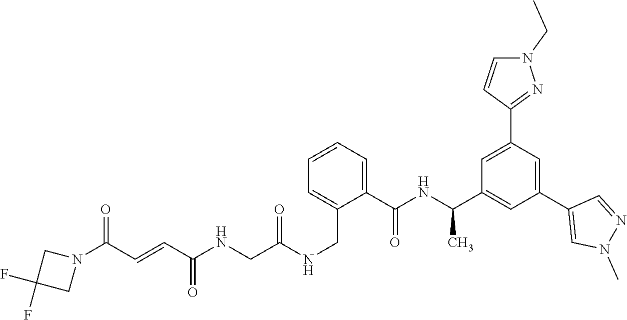 [SMILES: CCn1ccc(-c2cc(-c3cnn(C)c3)cc([C@@H](C)NC(=O)c3ccccc3CNC(=O)CNC(=O)/C=C/C(=O)N3CC(F)(F)C3)c2)n1]

White solid, 70% yield. ¹H NMR (400 MHZ, DMSO-d₆) δ 9.96 (s, 1H), 9.65 (s, 1H), 8.37 (d, J=8.1 Hz, 1H), 7.71 (s, 1H), 7.42 (s, 1H), 7.22 (s, 1H), 7.03 (s, 1H), 6.84 (dq, J=28.7, 7.2 Hz, 4H), 6.42 (d, J=7.3 Hz, 1H), 6.27 (d, J=2.3 Hz, 1H), 4.74-4.67 (m, 1H), 4.32 (t, J=12.3 Hz, 2H), 3.91 (t, J=12.4 Hz, 2H), 3.70 (q, J=7.2 Hz, 2H), 3.40 (s, 3H), 2.45 (d, J=7.7 Hz, 2H), 2.07-2.03 (m, 2H), 1.01 (d, J=7.0 Hz, 3H), 0.94 (t, J=7.3 Hz, 3H). ¹³C NMR (101 MHZ, DMSO-d₆) δ 170.44, 168.67, 162.19, 150.33, 146.11, 138.98, 137.65, 136.51, 134.61, 133.33, 132.36, 131.14, 129.87, 129.81, 129.12, 128.41, 127.70, 126.38, 122.42, 122.34, 120.84, 120.58, 103.00, 48.91, 46.81, 39.10, 35.00, 28.47, 23.02, 16.01. C₃₄H₃₆F₂N₈O₄, HRMS calculated for m/z [M+H]⁺: 659.2906 (calculated), 659.2911 (found).

Example 20 Synthesis of (R,E)-2-(3-(2-(4-(Bicyclo[1.1.1]pentan-1-ylamino)-4-oxobut-2-enoyl)hydrazineyl)-3-oxopropyl)-N-(1-(3-(1-ethyl-1H-pyrazol-3-yl)-5-(1-methyl-1 H-pyrazol-4-yl)phenyl)ethyl)benzamide

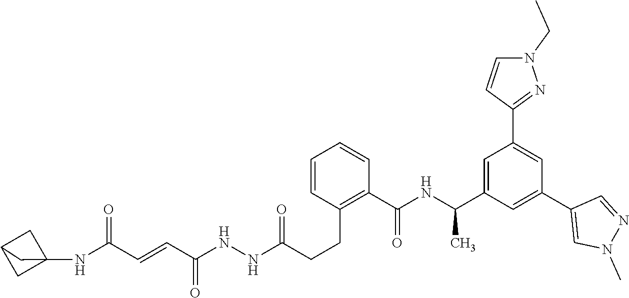 [SMILES: CCn1ccc(-c2cc(-c3cnn(C)c3)cc([C@@H](C)NC(=O)c3ccccc3CCC(=O)NNC(=O)/C=C/C(=O)NC34CC(C3)C4)c2)n1]

White solid, 58% yield. ¹H NMR (400 MHZ, DMSO-d₆) δ 9.94 (s, 1H), 9.62 (s, 1H), 8.51 (s, 1H), 8.42 (d, J=8.2 Hz, 1H), 7.76 (s, 1H), 7.47 (s, 1H), 7.38 (s, 1H), 7.35 (d, J=2.3 Hz, 1H), 7.27 (s, 1H), 7.08 (s, 1H), 6.94-6.83 (m, 4H), 6.40 (s, 2H), 6.31 (d, J=2.3 Hz, 1H), 4.79-4.72 (m, 1H), 3.75 (q, J=7.3 Hz, 2H), 3.45 (s, 3H), 2.51 (t, J=6.7 Hz, 2H), 2.10 (s, 1H), 2.00 (s, 1H), 1.59 (s, 6H), 1.06 (d, J=7.0 Hz, 3H), 0.99 (t, J=7.3 Hz, 3H). ¹³C NMR (101 MHz, DMSO-d₆) δ 170.48, 168.66, 163.87, 162.64, 150.34, 146.12, 139.00, 137.66, 136.52, 134.62, 134.49, 133.34, 131.14, 130.65, 129.88, 129.81, 128.42, 127.70, 126.38, 122.43, 122.35, 120.84, 120.58, 103.01, 52.73, 49.00, 48.91, 46.81, 39.10, 35.03, 28.48, 25.11, 23.02, 16.01. C₃₆H₄₀N₈O₄, HRMS calculated for m/z [M+H]⁺: 649.3251 (calculated), 649.3259 (found).

### Example 21 Synthesis of (R,E)-N-(1-(3-(1-ethyl-1H-pyrazol-3-yl)-5-(1-methyl-1H-pyrazol-4-yl)phenyl)ethyl)-2-(3-(2-(4-(3-fluoroazetidin-1-yl)-4-oxobut-2-enoyl)hydrazineyl)-3-oxopropyl)benzamide

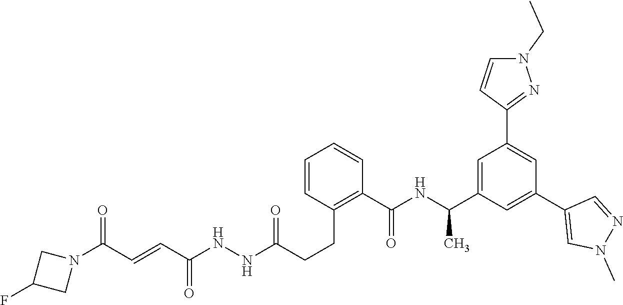 [SMILES: CCn1ccc(-c2cc(-c3cnn(C)c3)cc([C@@H](C)NC(=O)c3ccccc3CCC(=O)NNC(=O)/C=C/C(=O)N3CC(F)C3)c2)n1]

White solid, 78% yield. ¹H NMR (400 MHZ, DMSO-d₆) δ 10.35 (s, 1H), 10.04 (s, 1H), 8.79 (d, J=8.1 Hz, 1H), 8.12 (s, 1H), 7.84 (s, 1H), 7.75 (s, 1H), 7.71 (d, J=2.3 Hz, 1H), 7.64 (s, 1H), 7.45 (s, 1H), 7.28 (p, J=6.8 Hz, 3H), 7.21 (q, J=7.0 Hz, 1H), 6.81 (d, J=3.0 Hz, 1H), 6.68 (d, J=2.3 Hz, 1H), 5.42 (tt, J=6.2, 3.2 Hz, 1H), 5.27 (tt, J=6.2, 3.2 Hz, 2H), 5.16-5.09 (m, 1H), 4.55 (ddd, J=19.4, 9.8, 6.1 Hz, 1H), 4.37-4.16 (m, 2H), 4.11 (q, J=7.3 Hz, 2H), 3.93 (ddd, J=25.0, 12.1, 4.9 Hz, 1H), 2.87 (d, J=7.9 Hz, 2H), 2.46 (t, J=7.9 Hz, 2H), 1.43 (d, J=7.1 Hz, 3H), 1.35 (t, J=7.3 Hz, 3H). ¹³C NMR (101 MHz, DMSO-d₆) δ 170.49, 168.68, 164.03, 162.39, 150.34, 146.13, 138.99, 137.66, 136.51, 134.62, 133.34, 131.82, 131.14, 129.87, 129.81, 129.07, 128.43, 127.71, 126.38, 122.43, 122.36, 120.85, 120.59, 103.01, 83.85, 81.86, 58.36, 58.10, 56.27, 56.02, 48.92, 46.81, 39.09, 35.01, 28.48, 23.02, 16.00. C₃₄H₃₇FN₈O₄, HRMS calculated for m/z [M+H]⁺: 641.3000 (calculated), 641.3010 (found).

### Example 22 Synthesis of (R,E)-2-(3-(2-(4-(azetidin-1-yl)-4-oxobut-2-enoyl) hydrazineyl)-3-oxopropyl)-N-(1-(3-(1-ethyl-1H-pyrazol-3-yl)-5-(1-methyl-1H-pyrazol-4-yl)phenyl)ethyl)benzamide

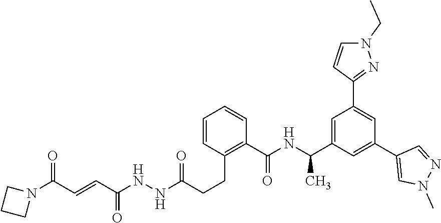 [SMILES: CCn1ccc(-c2cc(-c3cnn(C)c3)cc([C@@H](C)NC(=O)c3ccccc3CCC(=O)NNC(=O)/C=C/C(=O)N3CCC3)c2)n1]

White solid, 61% yield. ¹H NMR (400 MHZ, DMSO-d₆) δ 10.33 (s, 1H), 10.02 (s, 1H), 8.79 (d, J=8.0 Hz, 1H), 8.12 (s, 1H), 7.84 (s, 1H), 7.75 (s, 1H), 7.72 (d, J=2.2 Hz, 1H), 7.64 (d, J=2.1 Hz, 1H), 7.45 (s, 1H), 7.32-7.18 (m, 4H), 6.78 (d, J=1.7 Hz, 2H), 6.68 (d, J=2.3 Hz, 1H), 5.16-5.08 (m, 1H), 4.20 (t, J=7.7 Hz, 3H), 4.11 (q, J=7.3 Hz, 2H), 3.87 (t, J=7.7 Hz, 2H), 3.81 (s, 3H), 2.87 (d, J=7.9 Hz, 2H), 2.46 (t, J=7.2 Hz, 2H), 2.15 (p, J=7.6 Hz, 2H), 1.43 (d, J=7.0 Hz, 3H), 1.35 (t, J=7.3 Hz, 3H). ¹³C NMR (101 MHZ, DMSO-d₆) δ 170.52, 168.68, 163.53, 162.57, 150.33, 146.13, 138.99, 137.65, 136.51, 134.61, 133.33, 131.25, 131.15, 129.87, 129.82, 128.99, 128.42, 127.70, 126.38, 122.42, 122.35, 120.83, 120.58, 103.01, 50.44, 48.92, 48.29, 46.81, 39.09, 35.01, 28.47, 23.02, 16.01, 15.26. C₃₄H₃₈N₈O₄, HRMS calculated for m/z [M+H]⁺: 623.3094 (calculated), 623.3101 (found).

### Example 23 Synthesis of ethyl (E)-4-((2-((2-((1-(3-(1-ethyl-1H-pyrazol-3-yl)-5-(1-methyl-1H-pyrazol-4-yl)phenyl)cyclopropyl)carbamoyl)benzyl)amino)-2-oxoethyl)amino)-4-oxobut-2-enoate

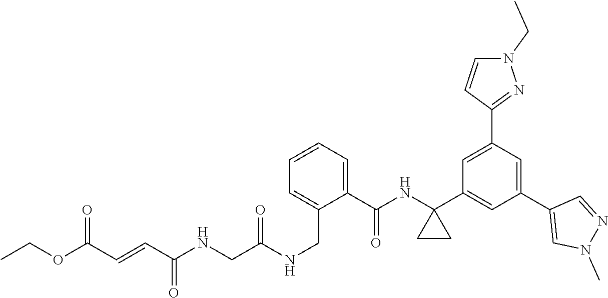 [SMILES: CCOC(=O)/C=C/C(=O)NCC(=O)NCc1ccccc1C(=O)NC1(c2cc(-c3cnn(C)c3)cc(-c3ccn(CC)n3)c2)CC1]

White solid, 71% yield. ¹H NMR (400 MHZ, DMSO-d₆) δ 9.14 (s, 1H), 8.83 (t, J=5.9 Hz, 1H), 8.35 (t, J=6.0 Hz, 1H), 8.11 (s, 1H), 7.83 (s, 1H), 7.70 (d, J=2.3 Hz, 1H), 7.68 (s, 1H), 7.52 (s, 1H), 7.43 (dd, J=7.8, 1.5 Hz, 1H), 7.37 (td, J=7.5, 1.5 Hz, 1H), 7.28 (dt, J=7.4, 3.8 Hz, 2H), 7.17 (s, 1H), 7.03 (d, J=15.5 Hz, 1H), 6.66 (d, J=2.3 Hz, 1H), 6.53 (d, J=15.6 Hz, 1H), 4.39 (d, J=5.9 Hz, 2H), 4.11 (qd, J=7.1, 3.6 Hz, 4H), 3.83 (d, J=5.9 Hz, 2H), 3.80 (s, 3H), 1.34 (t, J=7.3 Hz, 3H), 1.30 (d, J=2.1 Hz, 2H), 1.26 (d, J=3.1 Hz, 2H), 1.16 (t, J=7.1 Hz, 3H). ¹³C NMR (101 MHz, DMSO-d₆) δ 169.71, 168.96, 165.51, 163.78, 150.31, 144.74, 137.82, 137.43, 136.52, 136.24, 134.34, 133.22, 131.11, 130.16, 128.98, 128.41, 127.91, 127.77, 127.09, 122.49, 120.30, 119.82, 119.70, 102.97, 61.16, 46.81, 42.84, 39.07, 34.87, 18.62, 15.98, 14.46. C₃₄H₃₇N₇O₅, HRMS calculated for m/z [M+H]⁺: 624.2934 (calculated), 624.2956 (found).

### Example 24 Synthesis of (E)-2-((2-(4-(3,3-difluoroazetidin-1-yl)-4-oxobut-2-enamido)-acetamido)methyl)-N-(1-(3-(1-ethyl-1H-pyrazol-3-yl)-5-(1-methyl-1H-pyrazol-4-yl)phenyl)cyclopropyl)benzamide

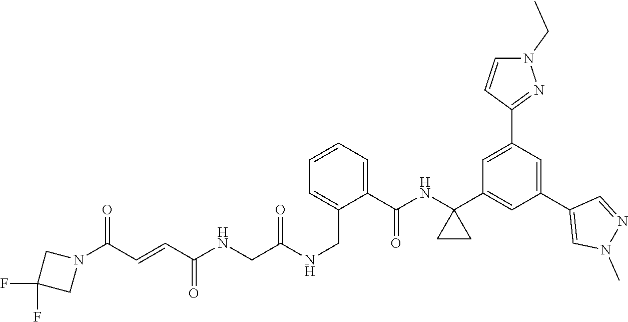 [SMILES: CCn1ccc(-c2cc(-c3cnn(C)c3)cc(C3(NC(=O)c4ccccc4CNC(=O)CNC(=O)/C=C/C(=O)N4CC(F)(F)C4)CC3)c2)n1]

White solid, 68% yield. ¹H NMR (400 MHZ, DMSO-d₆) δ 9.12 (s, 1H), 8.74 (t, J=6.0 Hz, 1H), 8.31 (t, J=6.0 Hz, 1H), 8.10 (s, 1H), 7.82 (s, 1H), 7.69 (s, 1H), 7.68 (s, 1H), 7.52 (s, 1H), 7.43 (d, J=7.5 Hz, 1H), 7.36 (d, J=7.4 Hz, 1H), 7.28 (d, J=7.1 Hz, 2H), 7.18 (s, 1H), 6.91 (d, J=15.2 Hz, 1H), 6.75 (d, J=15.2 Hz, 1H), 6.65 (s, 1H), 4.70 (t, J=12.4 Hz, 2H), 4.39 (d, J=5.9 Hz, 2H), 4.31 (t, J=12.5 Hz, 2H), 4.10 (q, J=7.3 Hz, 2H), 3.82 (d, J=5.8 Hz, 2H), 3.80 (s, 3H), 1.34 (t, J=7.3 Hz, 3H), 1.29 (s, 2H), 1.25 (s, 2H). ¹³C NMR (101 MHz, DMSO-d₆) δ 169.70, 169.05, 164.62, 164.21, 150.34, 144.75, 137.45, 136.53, 136.30, 134.81, 134.37, 133.24, 131.10, 130.15, 128.39, 127.98, 127.77, 127.10, 122.51, 120.37, 119.86, 119.74, 102.97, 46.81, 42.82, 39.08, 34.90, 18.59, 15.97. C₃₅H₃₆F₂N₈O₄, HRMS calculated for m/z [M+H]⁺: 671.2906 (calculated), 671.2915 (found).

### Example 25 Synthesis of (R,E)-2-((2-(4-(3,3-difluoroazetidin-1-yl)-4-oxobut-2-enamido)acetamido)methyl)-N-(1-(3-(1-ethyl-1H-pyrazol-3-yl)-5-(1-methyl-1H-pyrazol-4-yl)phenyl)ethyl)benzamide

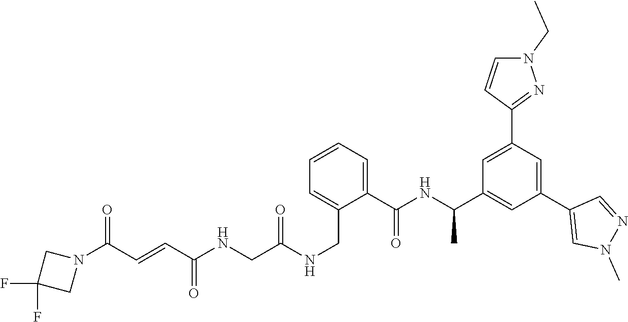 [SMILES: CCn1ccc(-c2cc(-c3cnn(C)c3)cc([C@@H](C)NC(=O)c3ccccc3CNC(=O)CNC(=O)/C=C/C(=O)N3CC(F)(F)C3)c2)n1]

White solid, 75% yield. ¹H NMR (400 MHz, DMSO-d₆) δ 8.86 (d, J=8.0 Hz, 1H), 8.73 (t, J=5.9 Hz, 1H), 8.30 (t, J=6.0 Hz, 1H), 8.10 (s, 1H), 7.82 (s, 1H), 7.74 (s, 1H), 7.70 (d, J=2.1 Hz, 1H), 7.62 (s, 1H), 7.44 (s, 1H), 7.39-7.32 (m, 2H), 7.28-7.23 (m, 2H), 6.90 (d, J=15.2 Hz, 1H), 6.75 (d, J=15.2 Hz, 1H), 6.67 (s, 1H), 5.15-5.07 (m, 1H), 4.70 (t, J=12.4 Hz, 2H), 4.36-4.27 (m, 4H), 4.11 (q, J=7.2 Hz, 2H), 3.81 (d, J=4.5 Hz, 5H), 1.44 (d, J=7.0 Hz, 3H), 1.34 (t, J=7.2 Hz, 3H). ¹³C NMR (101 MHz, DMSO-d₆) δ 169.08, 168.23, 164.63, 164.21, 150.32, 146.11, 137.32, 136.52, 136.33, 134.81, 134.65, 133.37, 131.11, 130.03, 128.40, 127.96, 127.91, 127.82, 127.06, 122.44, 122.24, 120.91, 120.61, 116.43, 103.02, 49.04, 46.81, 42.82, 39.09, 22.96, 16.00. C₃₄H₃₆F₂N₈O₄, HRMS calculated for m/z [M+H]⁺: 659.2906 (calculated), 659.2914 (found).

### Example 26. Additional Compounds

Using procedures similar to those described herein, the following compounds were also prepared.

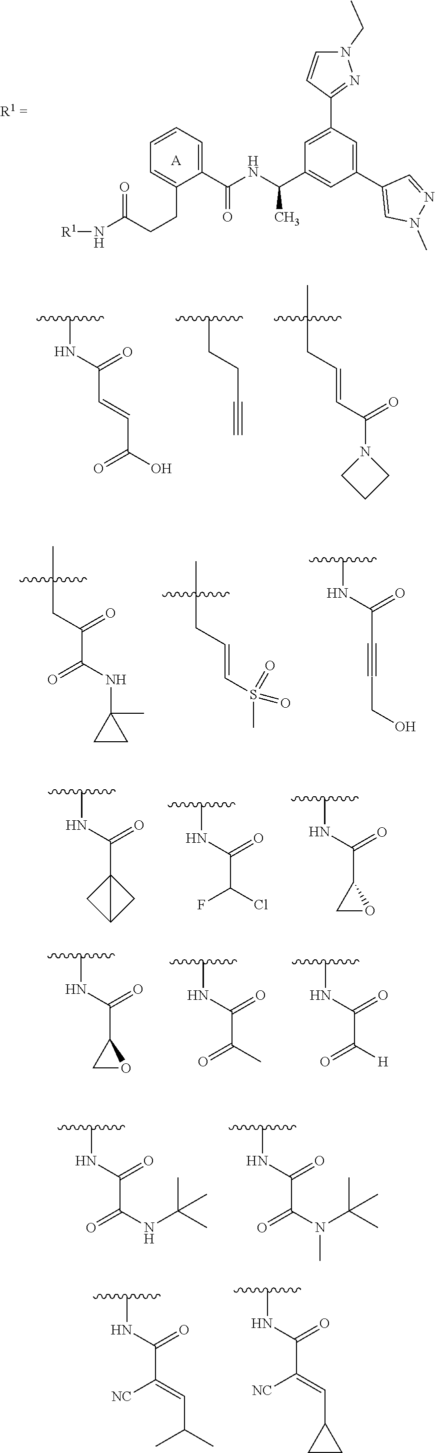

### Example 27. Synthesis of (R)-2-(3-(2-(bicyclo[1.1.1]pentane-1-carbonyl)hydrazineyl)-3-oxopropyl)-N-(1-(3-(1-ethyl-1H-pyrazol-3-yl)-5-(1-methyl-1H-pyrazol-4-yl)phenyl)ethyl)benzamide

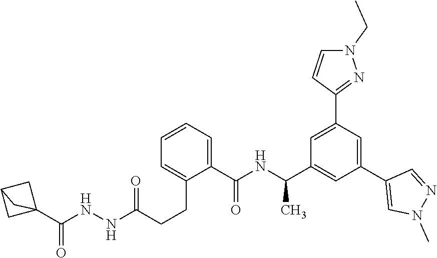 [SMILES: CCn1ccc(-c2cc(-c3cnn(C)c3)cc([C@@H](C)NC(=O)c3ccccc3CCC(=O)NNC(=O)C34CC(C3)C4)c2)n1]

White solid, 72% yield. ¹H NMR (400 MHZ, DMSO-d₆) δ 9.56 (s, 1H), 9.51 (s, 1H), 8.78 (d, J=8.2 Hz, 1H), 8.12 (s, 1H), 7.85-7.82 (m, 1H), 7.75 (s, 1H), 7.72 (d, J=2.3 Hz, 1H), 7.63 (s, 1H), 7.45 (s, 1H), 7.29 (s, 1H), 7.27 (s, 1H), 7.22-7.19 (m, 1H), 6.68 (s, 1H), 5.15-5.07 (m, 1H), 4.12 (q, J=7.3 Hz, 2H), 3.82 (s, 3H), 2.85 (td, J=7.5, 3.1 Hz, 2H), 2.40 (t, J=7.7 Hz, 2H), 1.91 (s, 6H), 1.43 (d, J=6.9 Hz, 3H), 1.35 (t, J=7.3 Hz, 3H). ¹³C NMR (101 MHz, DMSO-d₆) δ 170.97, 168.68, 168.08, 150.34, 146.15, 139.06, 137.69, 136.50, 134.61, 133.33, 131.14, 129.84, 129.79, 128.43, 127.65, 126.32, 122.43, 122.35, 120.84, 120.58, 103.00, 51.16, 48.90, 46.80, 43.33, 39.09, 35.11, 28.47, 27.28, 23.13, 23.00, 21.54, 21.48, 16.01. C₃₃H₃₇N₇O₃, HRMS calculated for m/z [M+H]⁺: 580.3036 (calculated), 580.3036 (found)

### Example 28. Synthesis of (R)-N-(1-(3-(1-ethyl-1H-pyrazol-3-yl)-5-(1-methyl-1H-pyrazol-4-yl)phenyl)ethyl)-2-(3-((3-fluoro-2-oxopropyl)amino)-3-oxopropyl)benzamide

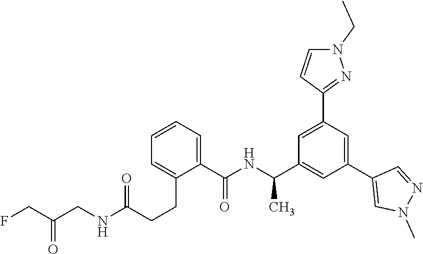 [SMILES: CCn1ccc(-c2cc(-c3cnn(C)c3)cc([C@@H](C)NC(=O)c3ccccc3CCC(=O)NCC(=O)CF)c2)n1]

White solid, 72% yield. ¹H NMR (400 MHZ, DMSO-d₆) δ 8.80 (d, J=8.3 Hz, 1H), 8.12 (s, 1H), 7.83 (s, 1H), 7.74 (s, 1H), 7.72 (s, 1H), 7.63 (s, 1H), 7.45 (s, 1H), 7.28 (d, J=7.2 Hz, 2H), 7.23 (d, J=6.9 Hz, 1H), 7.22-7.12 (m, 2H), 6.68 (s, 1H), 5.15-5.08 (m, 1H), 5.00 (d, J=46.5 Hz, 1H), 4.11 (q, J=7.3 Hz, 2H), 3.87 (d, J=5.3 Hz, 1H), 3.81 (s, 3H), 2.85 (t, J=7.8 Hz, 2H), 2.49-2.43 (m, 1H), 2.42 (d, J=4.3 Hz, 2H), 2.00 (s, 1H), 1.43 (d, J=7.2 Hz, 3H), 1.35 (t, J=7.3 Hz, 3H). ¹³C NMR (101 MHz, DMSO-d₆) δ 202.45, 172.58, 168.71, 150.33, 146.14, 140.96, 139.22, 137.66, 136.50, 134.60, 133.33, 132.88, 131.13, 130.49, 129.82, 129.75, 128.60, 128.42, 127.71, 126.29, 122.43, 122.34, 120.85, 120.58, 102.99, 85.62, 83.85, 48.92, 46.80, 45.71, 39.09, 36.71, 28.65, 23.00, 16.01. C₃₀H₃₃FN₆O₃, HRMS calculated for m/z [M+H]⁺: 545.2676 (calculated), 545.2682 (found)

### Example 29. Synthesis of (R)-2-((2-acrylamidoacetamido)methyl)-N-(1-(3-(1-methyl-1H-pyrazol-3-yl)-5-(1-methyl-1H-pyrazol-4-yl)phenyl)ethyl)benzamide

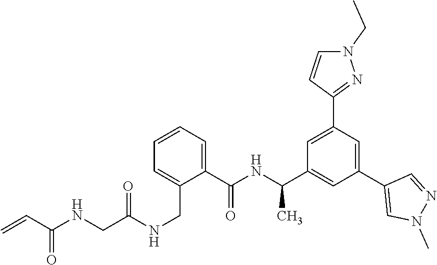 [SMILES: C=CC(=O)NCC(=O)NCc1ccccc1C(=O)N[C@H](C)c1cc(-c2cnn(C)c2)cc(-c2ccn(CC)n2)c1]

White solid, 84% yield. ¹H NMR (400 MHZ, DMSO-d₆) δ 8.87 (d, J=8.0 Hz, 1H), 8.33 (t, J=5.9 Hz, 1H), 8.26 (t, J=6.0 Hz, 1H), 8.11 (s, 1H), 7.83 (s, 1H), 7.74 (s, 1H), 7.66 (s, 1H), 7.62 (s, 1H), 7.44 (s, 1H), 7.39-7.32 (m, 2H), 7.29-7.23 (m, 2H), 6.67 (d, J=2.2 Hz, 1H), 6.24 (dd, J=17.1, 10.2 Hz, 1H), 6.04 (dd, J=17.2, 2.1 Hz, 1H), 5.53 (dd, J=10.2, 2.1 Hz, 1H), 5.15-5.07 (m, 1H), 4.34 (d, J=6.0 Hz, 2H), 3.82 (d, J=5.0 Hz, 6H), 3.77 (d, J=5.8 Hz, 2H), 1.43 (d, J=6.9 Hz, 3H). ¹³C NMR (101 MHz, DMSO-d₆) δ 169.45, 168.24, 165.47, 150.49, 146.15, 137.36, 136.49, 136.29, 134.51, 133.36, 132.71, 132.04, 130.04, 128.41, 127.95, 127.82, 127.07, 125.83, 122.42, 122.28, 120.89, 120.55, 103.15, 49.05, 42.62, 39.09, 22.96. C₂₉H₃₁N₇O₃, HRMS calculated for m/z [M+H]⁺: 526.2567 (calculated), 526.2572 (found)

### Example 30. Synthesis of (R)-N-(1-(3-(1-methyl-1H-pyrazol-3-yl)-5-(1-methyl-1H-pyrazol-4-yl)phenyl)ethyl)-2-((2-(vinylsulfonamido)acetamido)methyl)benzamide

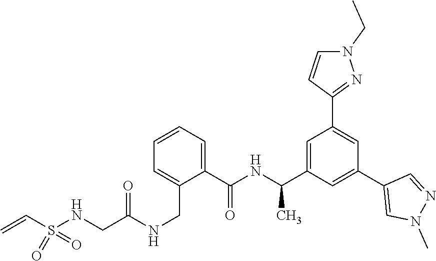 [SMILES: C=CS(=O)(=O)NCC(=O)NCc1ccccc1C(=O)N[C@H](C)c1cc(-c2cnn(C)c2)cc(-c2ccn(CC)n2)c1]

White solid, 77% yield. ¹H NMR (400 MHZ, DMSO-d₆) δ 8.89 (d, J=8.1 Hz, 1H), 8.26 (t, J=6.1 Hz, 1H), 8.11 (s, 1H), 7.83 (s, 1H), 7.75 (s, 1H), 7.67 (s, 1H), 7.62 (s, 1H), 7.44 (s, 1H), 7.37 (dd, J=17.4, 7.8 Hz, 2H), 7.27 (t, J=7.2 Hz, 2H), 6.67 (d, J=2.2 Hz, 1H), 6.63 (dd, J=16.5, 9.9 Hz, 1H), 5.95 (d, J=16.6 Hz, 1H), 5.82 (d, J=10.0 Hz, 1H), 5.16-5.08 (m, 1H), 4.34 (d, J=6.0 Hz, 2H), 3.82 (d, J=4.2 Hz, 6H), 1.44 (d, J=7.0 Hz, 3H). ¹³C NMR (101 MHz, DMSO-d₆) δ 168.65, 168.25, 150.48, 146.16, 137.26, 137.24, 136.49, 136.30, 134.51, 133.36, 132.72, 130.09, 128.43, 128.19, 127.87, 127.18, 125.95, 122.42, 122.30, 120.87, 120.56, 103.15, 49.07, 45.49, 40.56, 39.10, 22.97. C₂₈H₃₁N₇O₄S, HRMS calculated for m/z [M+H]⁺: 562.2236 (calculated), 562.2243 (found)

### Example 31. Synthesis of (R)-2-((2-(2-cyanoacetamido)acetamido)methyl)-N-(1-(3-(1-methyl-1H-pyrazol-3-yl)-5-(1-methyl-1H-pyrazol-4-yl)phenyl)ethyl)benzamide

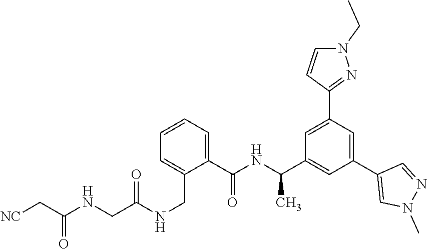 [SMILES: CCn1ccc(-c2cc(-c3cnn(C)c3)cc([C@@H](C)NC(=O)c3ccccc3CNC(=O)CNC(=O)CC#N)c2)n1]

White solid, 56% yield. ¹H NMR (400 MHZ, DMSO-d₆) δ 8.87 (d, J=8.0 Hz, 1H), 8.49 (t, J=5.8 Hz, 1H), 8.32 (t, J=6.0 Hz, 1H), 8.11 (s, 1H), 7.83 (s, 1H), 7.75 (s, 1H), 7.67 (s, 1H), 7.62 (s, 1H), 7.44 (s, 1H), 7.36 (dd, J=11.8, 7.4 Hz, 2H), 7.26 (dd, J=11.3, 7.5 Hz, 2H), 6.67 (d, J=2.2 Hz, 1H), 5.15-5.08 (m, 1H), 4.35 (d, J=6.0 Hz, 2H), 3.82 (d, J=4.1 Hz, 6H), 3.73 (d, J=5.7 Hz, 2H), 1.44 (d, J=6.9 Hz, 3H). ¹³C NMR (101 MHZ, DMSO-d₆) δ 168.93, 168.24, 163.17, 150.48, 146.15, 137.23, 136.49, 136.25, 134.51, 133.35, 132.71, 130.02, 128.41, 127.81, 127.74, 127.07, 122.42, 122.29, 120.89, 120.56, 116.54, 103.15, 49.05, 42.98, 39.09, 25.76, 22.95. C₂₉H₃₀N₈O₃, HRMS calculated for m/z [M+H]⁺: 539.2519 (calculated), 539.2530 (found)

### Example 32. Biological Assays

### FRET-Based PL^{pro} Enzymatic Assay

The SARS-CoV-2 PL^{pro} enzymatic assays were carried out as follows: The assay was assembled in 96-well plates with 100 μl of 200 nM PL^{Pro} protein in PL^{Pro} reaction buffer [50 mM Hepes (pH 7.5), 0.01% Triton X-100, and 5 mM DTT]. Then, 1 μl of testing compound at various concentrations was added to each well and incubated at 30° C. for 30 minutes. The enzymatic reaction was initiated by adding 1 μl of 1 mM FRET substrate (the final substrate concentration is 10 μM). The reaction was monitored in a Cytation 5 image reader with filters for excitation at 360/40 nm and emission at 460/40 nm at 30° C. for 1 hour. The initial velocity of the enzymatic reaction with and without testing compounds was calculated by linear regression for the first 15 minutes of the kinetic progress curve. The ICso values were calculated by plotting the initial velocity against various concentrations of testing compounds with a dose-response function in Prism 8 software. Data for representative compounds is provided in the following table.

### Cytotoxicity Assay in Vero E6 Cells

Evaluation of the cytotoxicity of compounds was carried out using the neutral red uptake assay (Repetto, G., et al., Nat Protoc 2008, 3, 1125-31). Briefly, 80,000 cells/ml of the tested cell lines were dispensed into 96-well cell culture plates at 100 μl per well. Twenty-four hours later, the growth medium was removed and washed with 150 μl of PBS buffer. Two hundred microliters of fresh serum-free medium containing serial diluted compounds was added to each well. After incubating for 2 days at 37° C., the medium was removed and replaced with 100 μl of DMEM medium containing neutral red (40 μg/ml) and incubated for 2 to 4 hours at 37° C. The amount of neutral red taken up was determined by measuring the absorbance at 540 nm using a Multiskan FC Microplate Photometer (Thermo Fisher Scientific). The CC₅₀ values were calculated from best-fit dose-response curves with variable slope in Prism 8. Data for representative compounds is provided in the following table.

TABLE

| Example | Compound | IC₅₀ | Vero CC₅₀ |
|---|---|---|---|
| 1 | 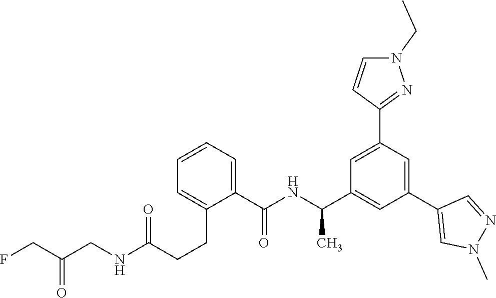 [SMILES: CCn1ccc(-c2cc(-c3cnn(C)c3)cc([C@@H](C)NC(=O)c3ccccc3CCC(=O)NCC(=O)CF)c2)n1] | C | 146 μM |
| 2 | 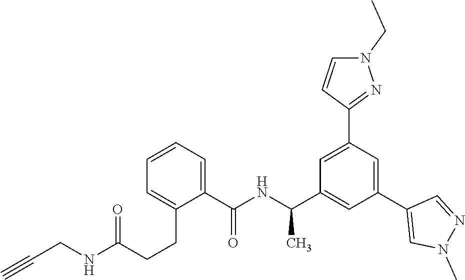 [SMILES: C#CCNC(=O)CCc1ccccc1C(=O)N[C@H](C)c1cc(-c2cnn(C)c2)cc(-c2ccn(CC)n2)c1] | B | 256 μM |
| 3 | 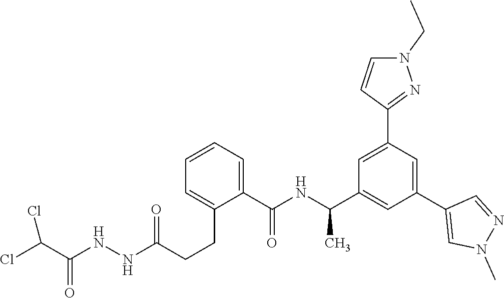 [SMILES: CCn1ccc(-c2cc(-c3cnn(C)c3)cc([C@@H](C)NC(=O)c3ccccc3CCC(=O)NNC(=O)C(Cl)Cl)c2)n1] | B | >500 μM |
| 4 | 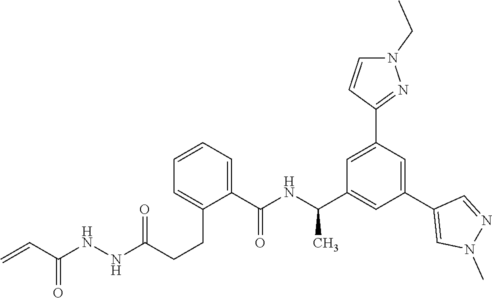 [SMILES: C=CC(=O)NNC(=O)CCc1ccccc1C(=O)N[C@H](C)c1cc(-c2cnn(C)c2)cc(-c2ccn(CC)n2)c1] | B | >500 μM |
| 5 | 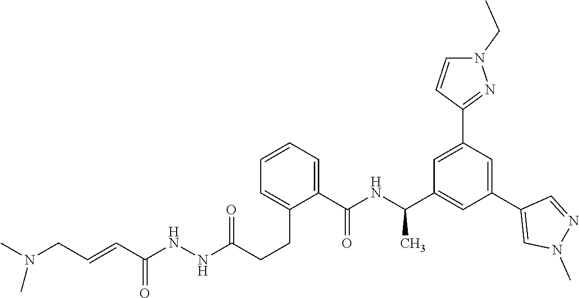 [SMILES: CCn1ccc(-c2cc(-c3cnn(C)c3)cc([C@@H](C)NC(=O)c3ccccc3CCC(=O)NNC(=O)/C=C/CN(C)C)c2)n1] | C | >500 μM |
| 6 | 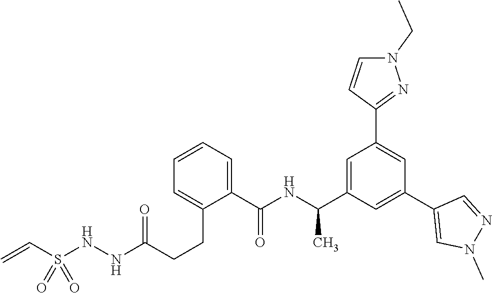 [SMILES: C=CS(=O)(=O)NNC(=O)CCc1ccccc1C(=O)N[C@H](C)c1cc(-c2cnn(C)c2)cc(-c2ccn(CC)n2)c1] | A | 31 μM |
| 7 | 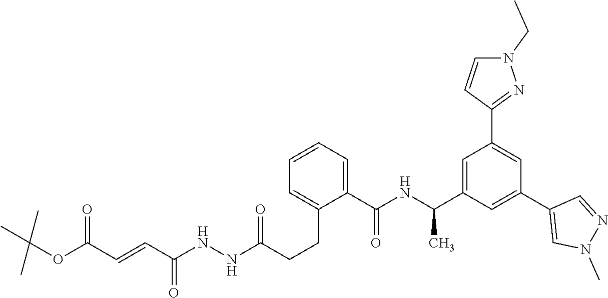 [SMILES: CCn1ccc(-c2cc(-c3cnn(C)c3)cc([C@@H](C)NC(=O)c3ccccc3CCC(=O)NNC(=O)/C=C/C(=O)OC(C)(C)C)c2)n1] | A | >250 μM |
| 8 | 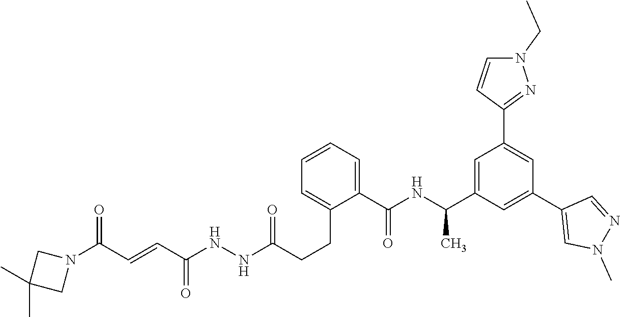 [SMILES: CCn1ccc(-c2cc(-c3cnn(C)c3)cc([C@@H](C)NC(=O)c3ccccc3CCC(=O)NNC(=O)/C=C/C(=O)N3CC(C)(C)C3)c2)n1] | A | >250 μM |
| 9 | 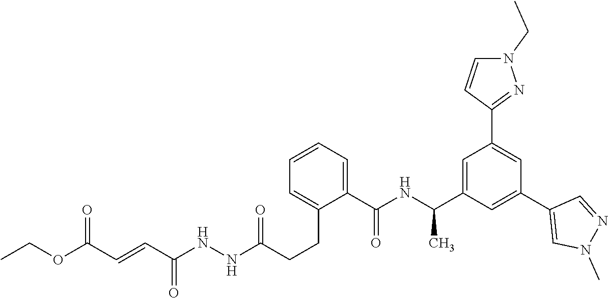 [SMILES: CCOC(=O)/C=C/C(=O)NNC(=O)CCc1ccccc1C(=O)N[C@H](C)c1cc(-c2cnn(C)c2)cc(-c2ccn(CC)n2)c1] | A | >250 μM |
| 10 | 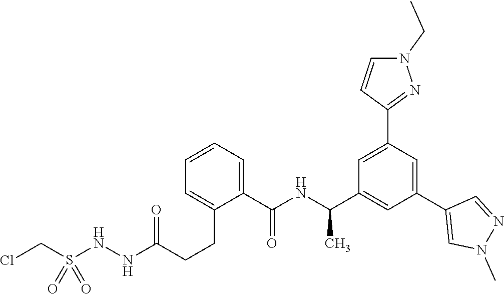 [SMILES: CCn1ccc(-c2cc(-c3cnn(C)c3)cc([C@@H](C)NC(=O)c3ccccc3CCC(=O)NNS(=O)(=O)CCl)c2)n1] | C | >250 μM |
| 11 | 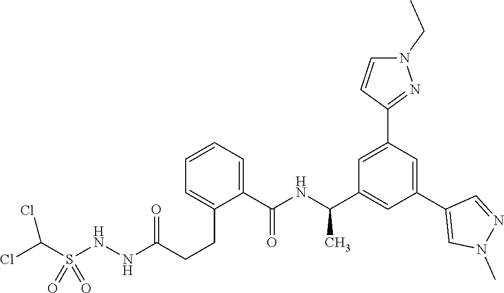 [SMILES: CCn1ccc(-c2cc(-c3cnn(C)c3)cc([C@@H](C)NC(=O)c3ccccc3CCC(=O)NNS(=O)(=O)C(Cl)Cl)c2)n1] | C | >250 μM |
| 12 | 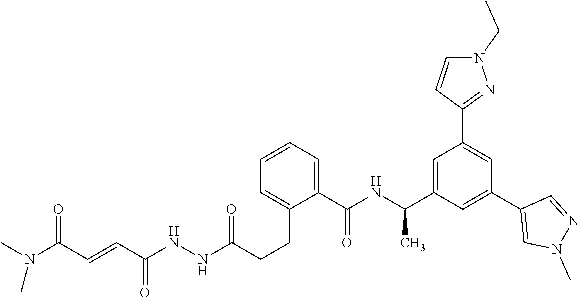 [SMILES: CCn1ccc(-c2cc(-c3cnn(C)c3)cc([C@@H](C)NC(=O)c3ccccc3CCC(=O)NNC(=O)/C=C/C(=O)N(C)C)c2)n1] | A | >500 μM |
| 13 | 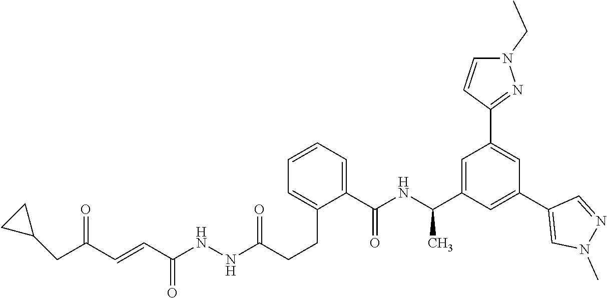 [SMILES: CCn1ccc(-c2cc(-c3cnn(C)c3)cc([C@@H](C)NC(=O)c3ccccc3CCC(=O)NNC(=O)/C=C/C(=O)CC3CC3)c2)n1] | A | >250 μM |
| 14 | 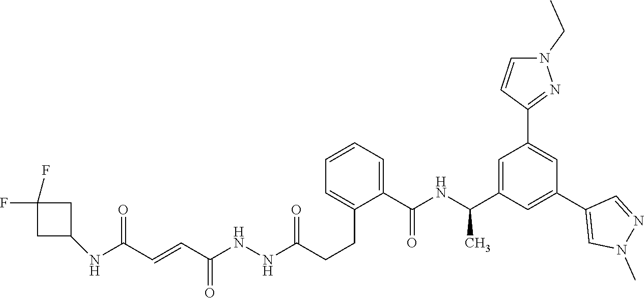 [SMILES: CCn1ccc(-c2cc(-c3cnn(C)c3)cc([C@@H](C)NC(=O)c3ccccc3CCC(=O)NNC(=O)/C=C/C(=O)NC3CC(F)(F)C3)c2)n1] | A | >250 μM |
| 15 | 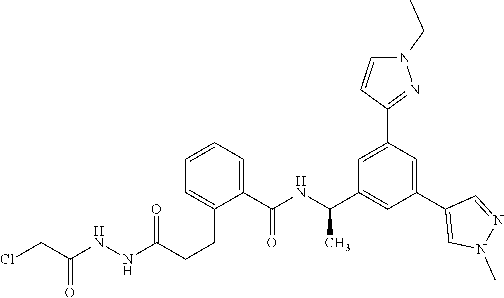 [SMILES: CCn1ccc(-c2cc(-c3cnn(C)c3)cc([C@@H](C)NC(=O)c3ccccc3CCC(=O)NNC(=O)CCl)c2)n1] | A | >250 μM |
| 16 | 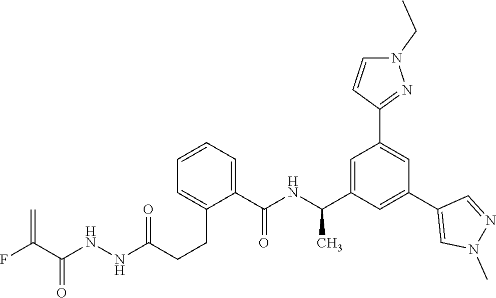 [SMILES: C=C(F)C(=O)NNC(=O)CCc1ccccc1C(=O)N[C@H](C)c1cc(-c2cnn(C)c2)cc(-c2ccn(CC)n2)c1] | C | >250 μM |
| 17 | 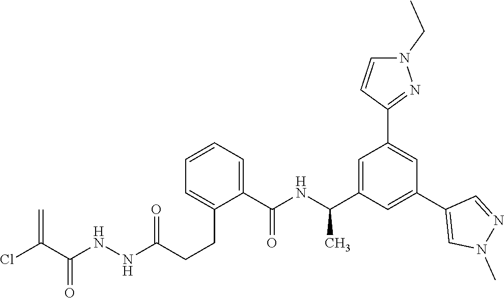 [SMILES: C=C(Cl)C(=O)NNC(=O)CCc1ccccc1C(=O)N[C@H](C)c1cc(-c2cnn(C)c2)cc(-c2ccn(CC)n2)c1] | B | 33 μM |
| 18 | 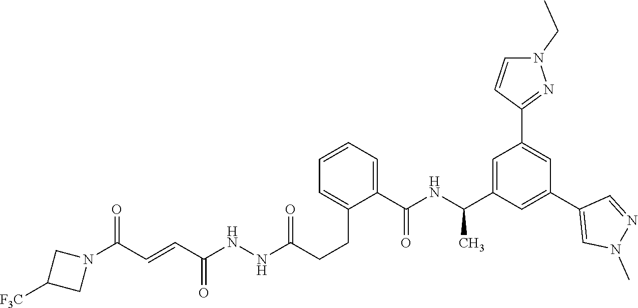 [SMILES: CCn1ccc(-c2cc(-c3cnn(C)c3)cc([C@@H](C)NC(=O)c3ccccc3CCC(=O)NNC(=O)/C=C/C(=O)N3CC(C(F)(F)F)C3)c2)n1] | A | >500 μM |
| 19 | 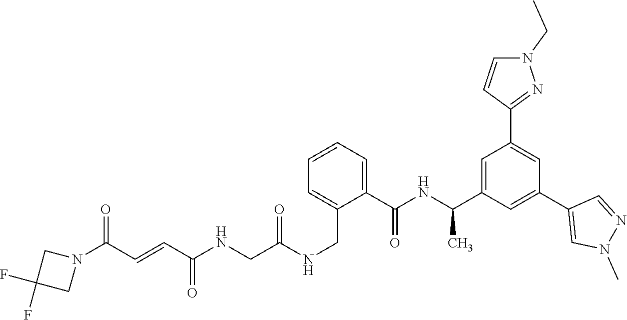 [SMILES: CCn1ccc(-c2cc(-c3cnn(C)c3)cc([C@@H](C)NC(=O)c3ccccc3CNC(=O)CNC(=O)/C=C/C(=O)N3CC(F)(F)C3)c2)n1] | A | >250 μM |
| 20 | 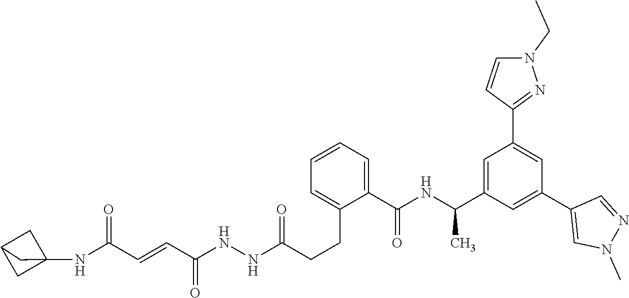 [SMILES: CCn1ccc(-c2cc(-c3cnn(C)c3)cc([C@@H](C)NC(=O)c3ccccc3CCC(=O)NNC(=O)/C=C/C(=O)NC34CC(C3)C4)c2)n1] | A | >250 μM |
| 21 | 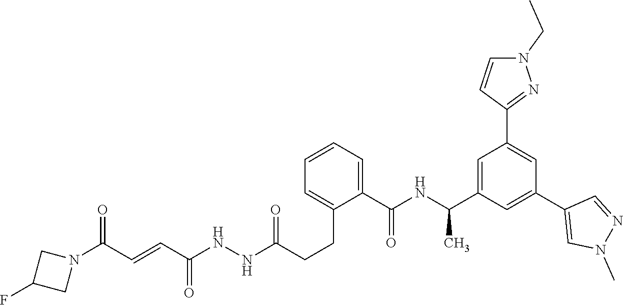 [SMILES: CCn1ccc(-c2cc(-c3cnn(C)c3)cc([C@@H](C)NC(=O)c3ccccc3CCC(=O)NNC(=O)/C=C/C(=O)N3CC(F)C3)c2)n1] | A | >500 μM |
| 22 | 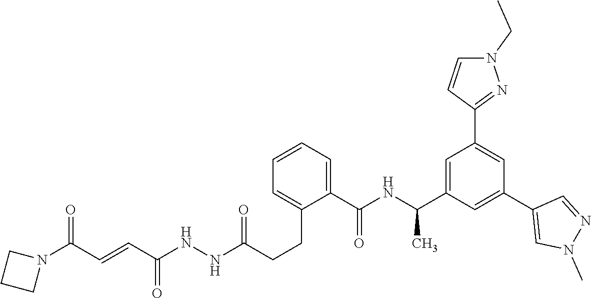 [SMILES: CCn1ccc(-c2cc(-c3cnn(C)c3)cc([C@@H](C)NC(=O)c3ccccc3CCC(=O)NNC(=O)/C=C/C(=O)N3CCC3)c2)n1] | A | >250 μM |
| 23 | 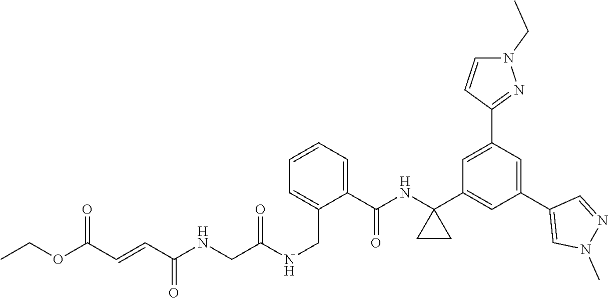 [SMILES: CCOC(=O)/C=C/C(=O)NCC(=O)NCc1ccccc1C(=O)NC1(c2cc(-c3cnn(C)c3)cc(-c3ccn(CC)n3)c2)CC1] | B | >500 μM |
| 24 | 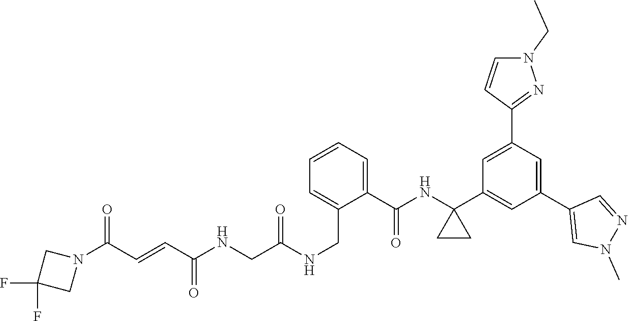 [SMILES: CCn1ccc(-c2cc(-c3cnn(C)c3)cc(C3(NC(=O)c4ccccc4CNC(=O)CNC(=O)/C=C/C(=O)N4CC(F)(F)C4)CC3)c2)n1] | B | >500 μM |
| 25 | 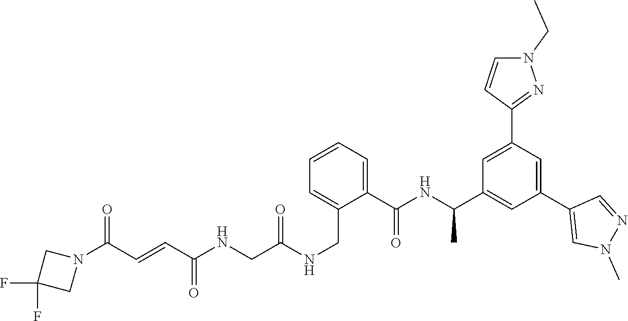 [SMILES: CCn1ccc(-c2cc(-c3cnn(C)c3)cc([C@@H](C)NC(=O)c3ccccc3CNC(=O)CNC(=O)/C=C/C(=O)N3CC(F)(F)C3)c2)n1] | A | >500 μM |
| 27 | 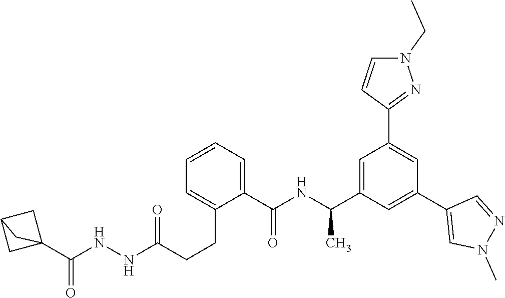 [SMILES: CCn1ccc(-c2cc(-c3cnn(C)c3)cc([C@@H](C)NC(=O)c3ccccc3CCC(=O)NNC(=O)C34CC(C3)C4)c2)n1] | C | >250 μM |
| 28 | 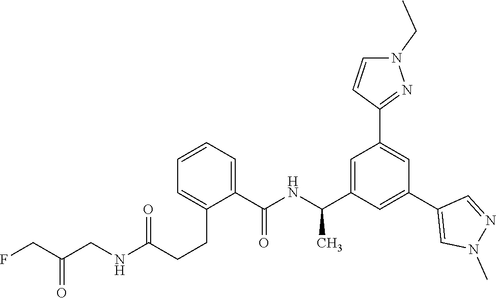 [SMILES: CCn1ccc(-c2cc(-c3cnn(C)c3)cc([C@@H](C)NC(=O)c3ccccc3CCC(=O)NCC(=O)CF)c2)n1] | C | >250 μM |
| 29 | 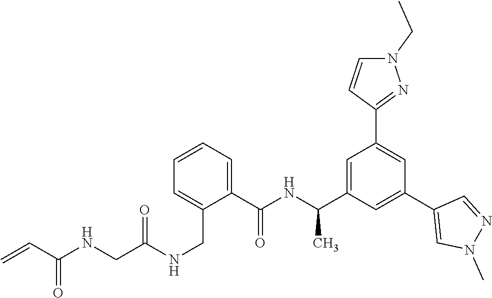 [SMILES: C=CC(=O)NCC(=O)NCc1ccccc1C(=O)N[C@H](C)c1cc(-c2cnn(C)c2)cc(-c2ccn(CC)n2)c1] | C | >500 μM |
| 30 | 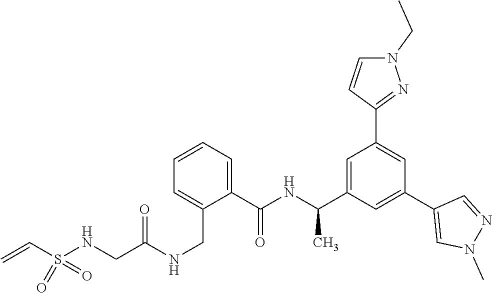 [SMILES: C=CS(=O)(=O)NCC(=O)NCc1ccccc1C(=O)N[C@H](C)c1cc(-c2cnn(C)c2)cc(-c2ccn(CC)n2)c1] | A | >250 μM |
| 31 | 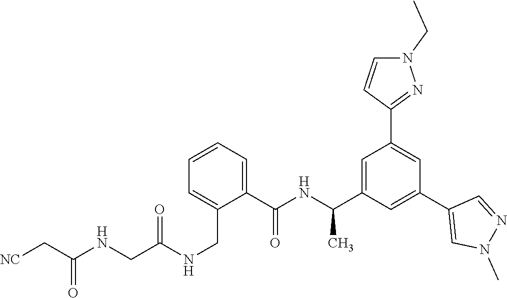 [SMILES: CCn1ccc(-c2cc(-c3cnn(C)c3)cc([C@@H](C)NC(=O)c3ccccc3CNC(=O)CNC(=O)CC#N)c2)n1] | C | >500 μM |

A: IC₅₀ ≤ 1 μM;
B: 1 μM < IC₅₀ ≤ 5 μM;
C: IC₅₀ > 5 μM.

### Example 33. The following illustrate representative pharmaceutical dosage forms, containing a compound of formula I (‘Compound X’), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | |
| (pH adjustment to 7.0-7.5) | q.s. |
| Water for injection | q.s. ad 1 mL |

| (v) Injection 2 (10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | |
| (pH adjustment to 7.0-7.5) | q.s. |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula (I): 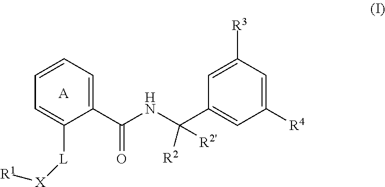
or a salt thereof, wherein:
R¹ is (C₁-C₆)alkyl, (C₂-C₆)alkenyl, (C₂-C₆)alkynyl, or —NR^{a}R^{b}, wherein any (C₁-C₆)alkyl, (C₂-C₆)alkenyl, and (C₂-C₆)alkynyl is optionally substituted with one or more groups independently selected from the group consisting of hydroxy, halo, cyano, oxo (═O), (C₁-C₆)alkoxy, —NR^{a}R^{b}, —C(═O)NR^{s}R^{t}, —C(═O)C(═O)NR^{s}R^{t}, S(═O)ₙR^{e}, and (C₃-C₆)cycloalkyl;
L is (C₁-C₃)alkylene that is optionally substituted with one or more groups independently selected from fluoro and chloro;
ring A is optionally substituted with one or more groups independently selected from the group consisting of halo, hydroxy, cyano, carboxy, (C₁-C₆)alkyl, (C₁-C₆)alkoxy, (C₁-C₆)alkoxycarbonyl, and (C₁-C₆)alkanoyloxy, wherein any (C₁-C₆)alkyl, (C₁-C₆)alkoxy, (C₁-C₆)alkoxycarbonyl, and (C₁-C₆)alkanoyloxy is optionally substituted with one or more groups independently selected from the group consisting of halo;
R² is H or (C₁-C₃)alkyl that is optionally substituted with one or more groups independently selected from halo and deuterium; and R^{2′} is H or (C₁-C₃)alkyl that is optionally substituted with one or more groups independently selected from halo and deuterium; or R² and R^{2′} taken together with the carbon to which they are attached form a (C₃-C₆)cycloalkyl;
R³ is a 5-membered heteroaryl that is optionally substituted with one or more groups independently selected from the group consisting of halo, hydroxy, cyano, (C₁-C₆)alkyl, and (C₁-C₆)alkoxy, wherein any (C₁-C₆)alkyl, and (C₁-C₆)alkoxy is optionally substituted with one or more groups independently selected from the group consisting of halo; and
R⁴ is a 5-membered heteroaryl that is optionally substituted with one or more groups independently selected from the group consisting of halo, hydroxy, cyano, (C₁-C₆)alkyl, and (C₁-C₆)alkoxy, wherein any (C₁-C₆)alkyl, and (C₁-C₆)alkoxy is optionally substituted with one or more groups independently selected from the group consisting of halo;
X is —NH—C(═O)— or —C(═O)—NH—;
R^{a} is H or (C₁-C₆)alkyl;
R^{b} is (C₁-C₆)alkyl, (C₂-C₆)alkenyl, (C₂-C₆)alkynyl, —C(═O)R^{c}, or S(═O)ₙR^{d}, wherein any (C₁-C₆)alkyl, (C₂-C₆)alkenyl, and (C₂-C₆)alkynyl is optionally substituted with one or more groups independently selected from the group consisting of halo, hydroxy, cyano, (C₁-C₆)alkoxy, —NR^{u}R^{v}, and (C₃-C₆)cycloalkyl;
R^{c} is H, (C₁-C₆)alkyl, (C₂-C₆)alkenyl, (C₂-C₆)alkynyl, 3-6 membered heterocycle, (C₁-C₆)alkanoyl, or (C₃-C₆)cycloalkyl, wherein any (C₁-C₆)alkyl, (C₂-C₆)alkenyl, (C₂-C₆)alkynyl, 3-6 membered heterocycle, (C₁-C₆)alkanoyl, and (C₃-C₆)cycloalkyl is optionally substituted with one or more groups independently selected from the group consisting of halo, hydroxy, cyano, (C₁-C₆)alkoxy, (C₁-C₆)alkoxycarbonyl, (C₁-C₆)alkanoyloxy, (C₃-C₆)cycloalkyl, oxo (═O), carboxy, —NR^{w}R^{x}, —C(═O)NR^{w}R^{x}, and (C₃-C₆)cycloalkyl;
R^{d} is (C₁-C₆)alkyl, (C₂-C₆)alkenyl, (C₂-C₆)alkynyl, or (C₃-C₆)cycloalkyl, wherein any (C₁-C₆)alkyl, (C₂-C₆)alkenyl, (C₂-C₆)alkynyl, and (C₃-C₆)cycloalkyl is optionally substituted with one or more groups independently selected from the group consisting of hydroxy, halo, cyano, (C₁-C₆)alkoxy, (C₁-C₆)alkoxycarbonyl, (C₁-C₆)alkanoyloxy, oxo (═O), —NR^{y}R_{z}, carboxy, —C(═O)NR^{y}R^{z}, and (C₃-C₆)cycloalkyl;
R^{e} is (C₁-C₆)alkyl, (C₂-C₆)alkenyl, or (C₂-C₆)alkynyl, wherein any (C₁-C₆)alkyl, (C₂-C₆)alkenyl, and (C₂-C₆)alkynyl is optionally substituted with one or more groups independently selected from the group consisting of hydroxy, halo, cyano, (C₁-C₆)alkoxy, (C₁-C₆)alkoxycarbonyl, (C₁-C₆)alkanoyloxy, —NR^{y}R^{z}, carboxy, —C(═O)NR^{y}R^{z}, and (C₃-C₆)cycloalkyl;
each R^{s} and R^{t} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl, wherein any (C₁-C₆)alkyl, (C₃-C₆)cycloalkyl, (C₃-C₆)cycloalkyl(C₁-C₆)alkyl, and (C₁-C₆)alkanolyl is optionally substituted with one or more groups independently selected from (C₁-C₆)alkyl and halo; or R^{s} and R^{t} together with the nitrogen to which they are attached form a aziridino, azetidino, morpholino, piperazino, pyrrolidino or piperidino, which a aziridino, azetidino, morpholino, piperazino, pyrrolidino and piperidino,is optionally substituted with one or more groups independently selected from halo and (C₁-C₆)alkyl that is optionally substituted with one or more groups independently selected from halo;
each R^{u} and R^{v} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl, wherein any (C₁-C₆)alkyl, (C₃-C₆)cycloalkyl, (C₃-C₆)cycloalkyl(C₁-C₆)alkyl, and (C₁-C₆)alkanolyl is optionally substituted with one or more groups independently selected from (C₁-C₆)alkyl and halo; or R^{u} and R^{v} together with the nitrogen to which they are attached form a aziridino, azetidino, morpholino, piperazino, pyrrolidino or piperidino, which a aziridino, azetidino, morpholino, piperazino, pyrrolidino and piperidino,is optionally substituted with one or more groups independently selected from halo and (C₁-C₆)alkyl that is optionally substituted with one or more groups independently selected from halo;
each R^{w} and R^{x} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl, wherein any (C₁-C₆)alkyl, (C₃-C₆)cycloalkyl, (C₃-C₆)cycloalkyl(C₁-C₆)alkyl, and (C₁-C₆)alkanolyl is optionally substituted with one or more groups independently selected from (C₁-C₆)alkyl and halo; or R^{w} and R^{x} together with the nitrogen to which they are attached form a aziridino, azetidino, morpholino, piperazino, pyrrolidino or piperidino, which a aziridino, azetidino, morpholino, piperazino, pyrrolidino and piperidino,is optionally substituted with one or more groups independently selected from halo and (C₁-C₆)alkyl that is optionally substituted with one or more groups independently selected from halo;
each R^{y} and R^{z} is independently selected from the group consisting of H, (C₁-C₆)alkyl, (C₃-C₆)cycloalkyl, (C₃-C₆)cycloalkyl(C₁-C₆)alkyl, and (C₁-C₆)alkanolyl, wherein any (C₁-C₆)alkyl, (C₃-C₆)cycloalkyl, (C₃-C₆)cycloalkyl (C₁-C₆)alkyl, and (C₁-C₆)alkanolyl is optionally substituted with one or more groups independently selected from (C₁-C₆)alkyl and halo; or R^{y} and R^{z} together with the nitrogen to which they are attached form a aziridino, azetidino, morpholino, piperazino, pyrrolidino or piperidino, which a aziridino, azetidino, morpholino, piperazino, pyrrolidino and piperidino,is optionally substituted with one or more groups independently selected from halo and (C₁-C₆)alkyl that is optionally substituted with one or more groups independently selected from halo; and
n is 0, 1, or 2.

2. The compound or salt of claim 1, wherein R¹ is —NR^{a}R^{b}.

3. The compound or salt of claim 1, wherein R¹ is selected from the group consisting of:

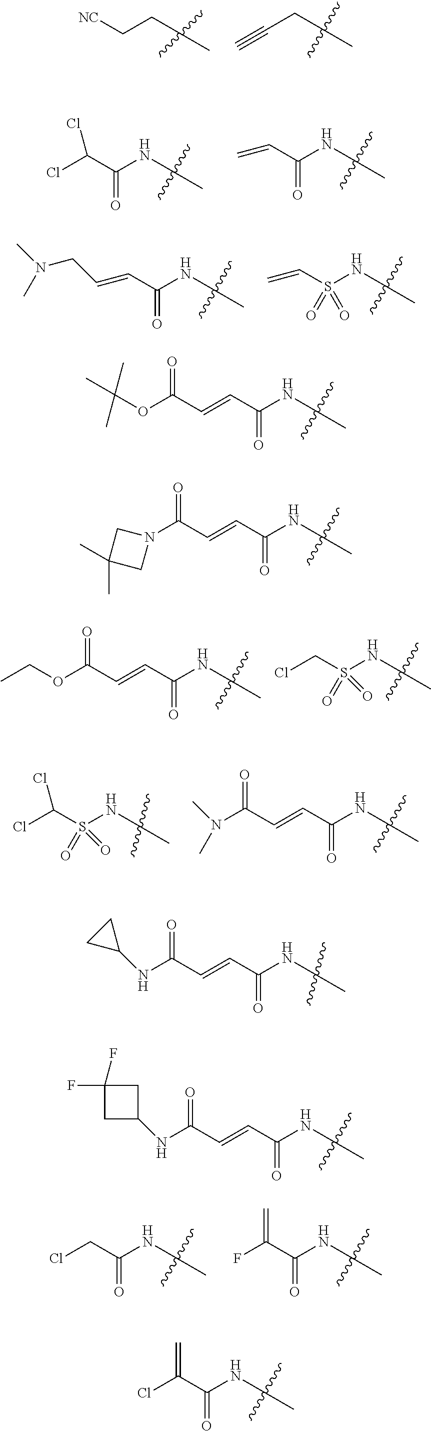

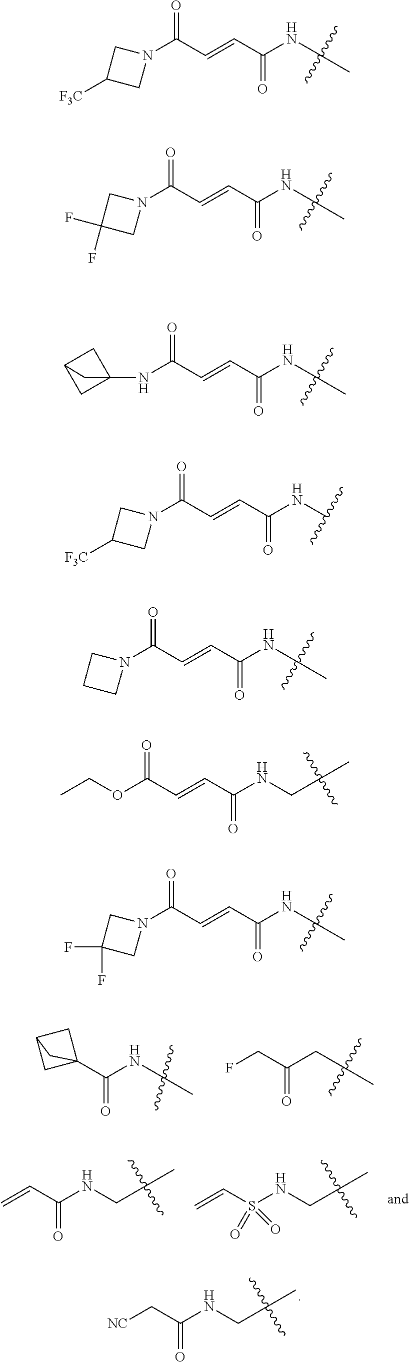

4. The compound or salt of claim 1, wherein L is —CH₂CH₂—.

5. The compound or salt of claim 1, wherein X is —NH—C(═O)—.

6. The compound or salt of claim 1, wherein X is —C(═O)—NH—.

7. The compound or salt of claim 1, wherein R³ is a 5-membered heteroaryl that comprises two nitrogen atoms that is optionally substituted with one or more groups independently selected from the group consisting of (C₁-C₆)alkyl.

8. The compound or salt of claim 1, wherein R³ is:

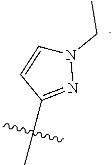 [SMILES: *c1ccn(CC)n1]

9. The compound or salt of claim 1, wherein R⁴ is a 5-membered heteroaryl that comprises two nitrogen atoms that is optionally substituted with one or more groups independently selected from the group consisting of (C₁-C₆)alkyl.

10. The compound or salt of claim 1, wherein R⁴ is:

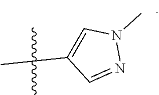 [SMILES: *c1cnn(C)c1]

11. The compound or salt of claim 1, wherein R^{a} is H.

12. The compound or salt of claim 1, wherein R^{b} is —C(═O)R^{c}.

13. The compound or salt of claim 1, wherein R^{b} is S(═O)ₙR^{d}.

14. The compound or salt of claim 1, which is a compound of formula (Ia): 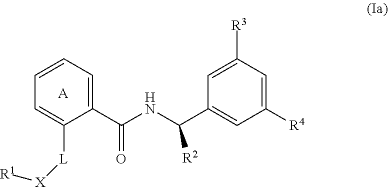
or a salt thereof.

15. The compound or salt of claim 1, which is a compound of formula (Ic): 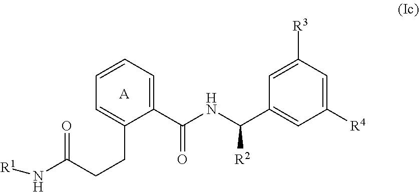
or a salt thereof.

16. The compound or salt of claim 1, which is a compound of formula (Ie): 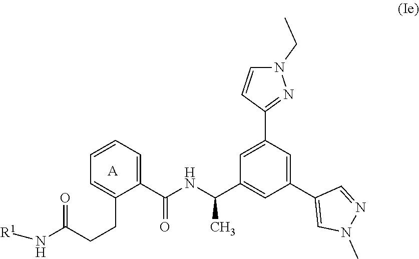
or a salt thereof.

17. The compound or salt of claim 1, wherein the group -L-X—R¹ has a structure selected from the group consisting of:

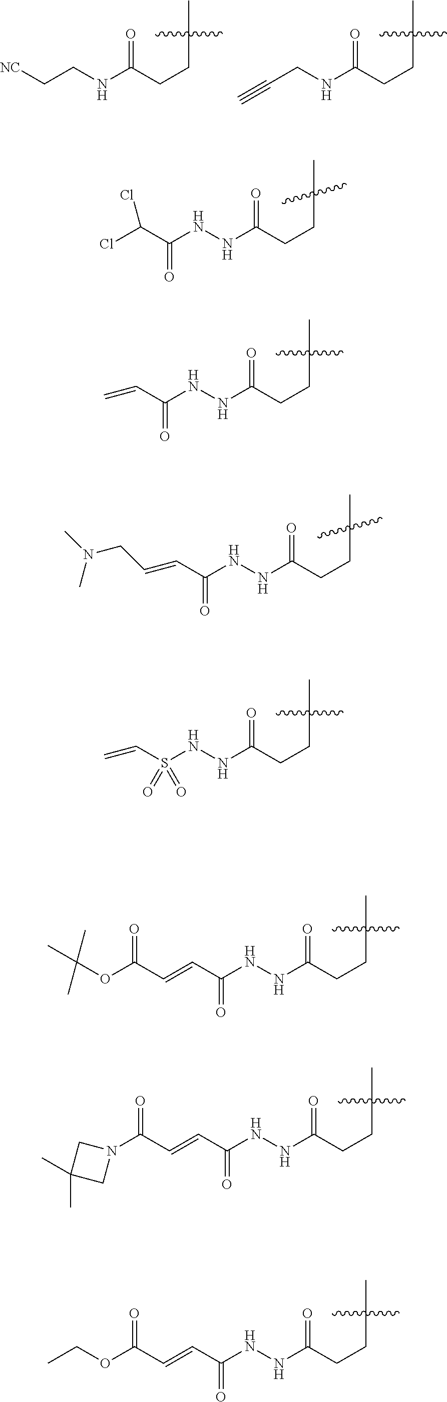

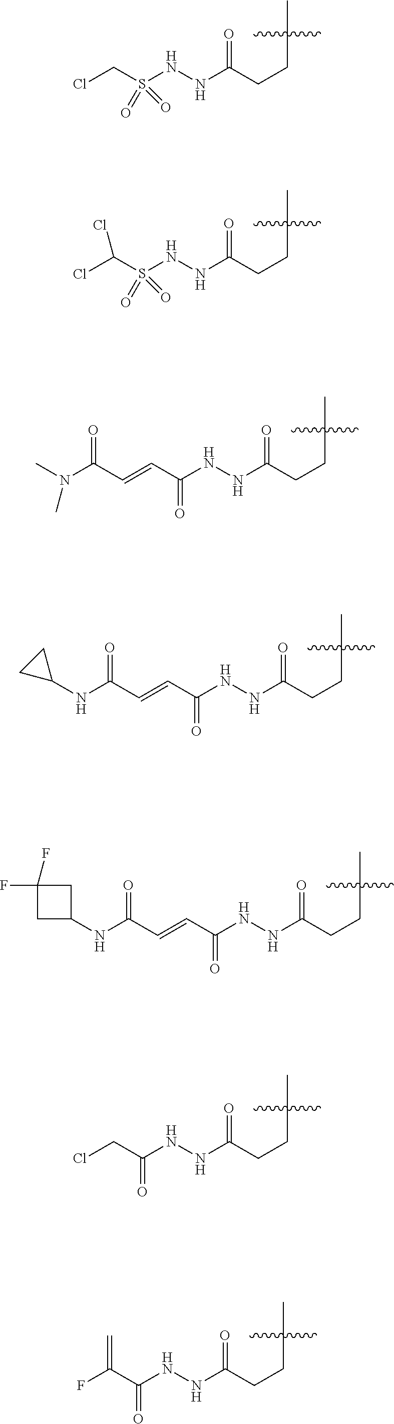

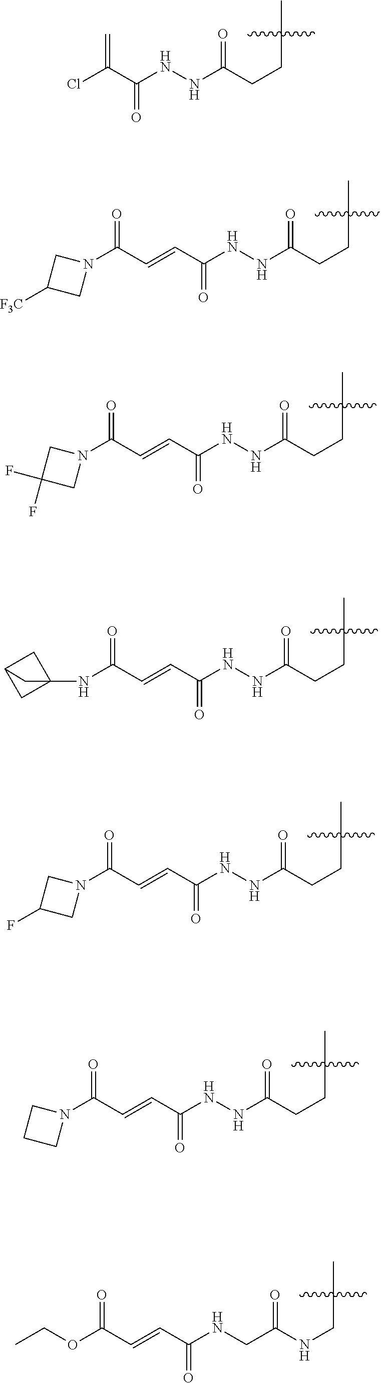

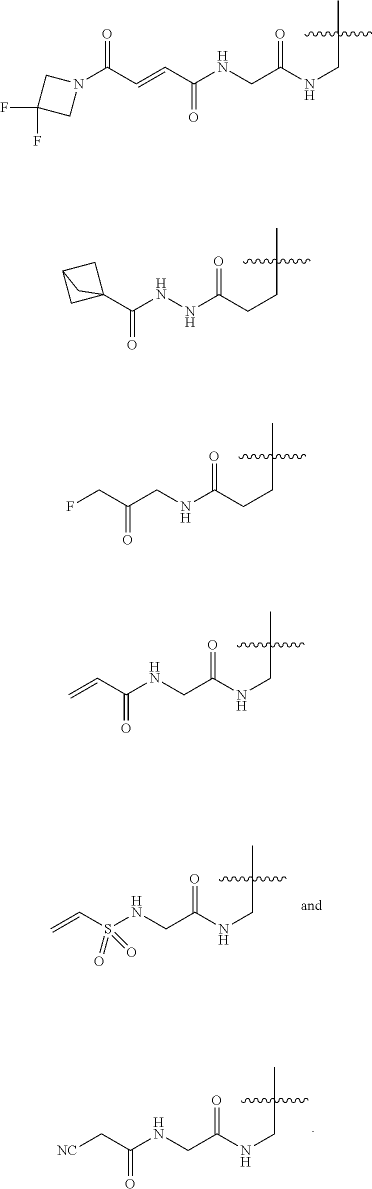 [SMILES: *CNC(=O)CNC(=O)/C=C/C(=O)N1CC(F)(F)C1 ; *CCC(=O)NNC(=O)C12CC(C1)C2 ; *CCC(=O)NCC(=O)CF ; *CNC(=O)CNC(=O)C=C ; *CNC(=O)CNS(=O)(=O)C=C ; *CNC(=O)CNC(=O)CC#N]

18. A compound or salt of claim 1 that is selected from the group consisting of: 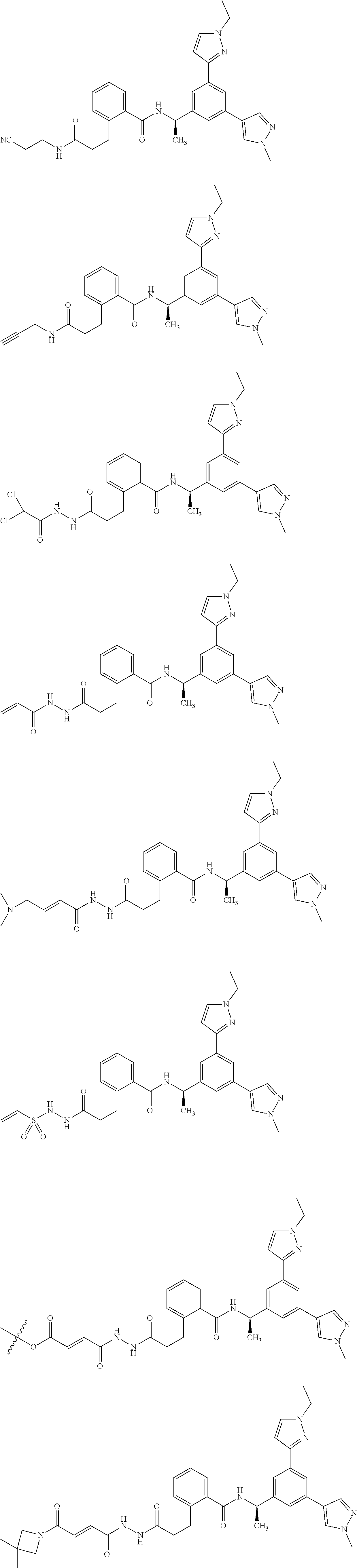 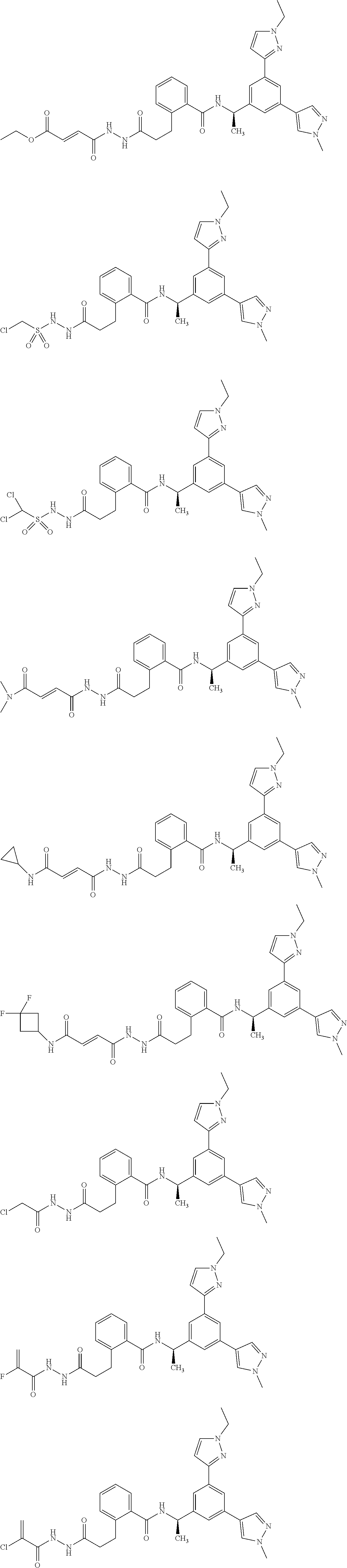 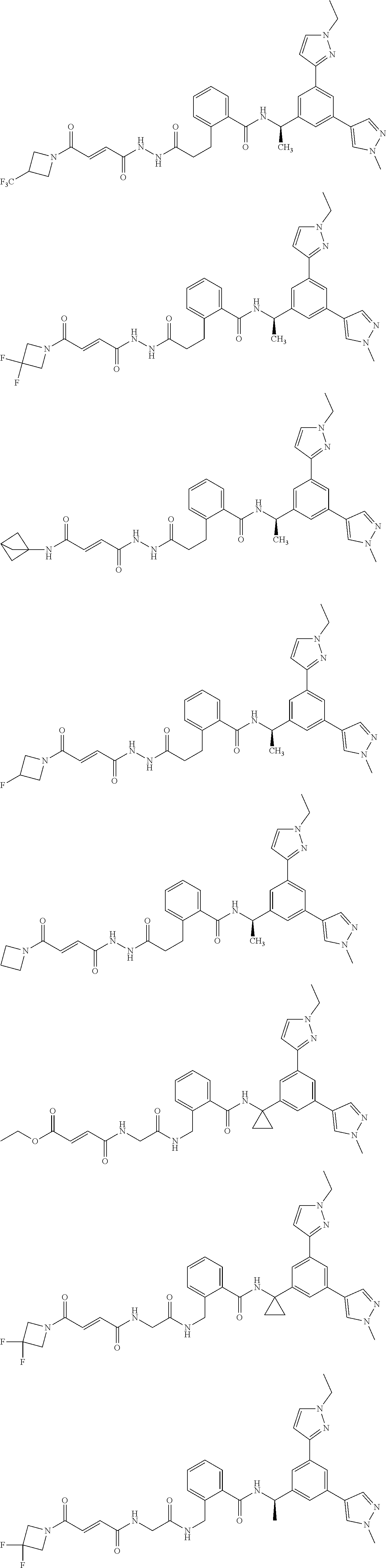 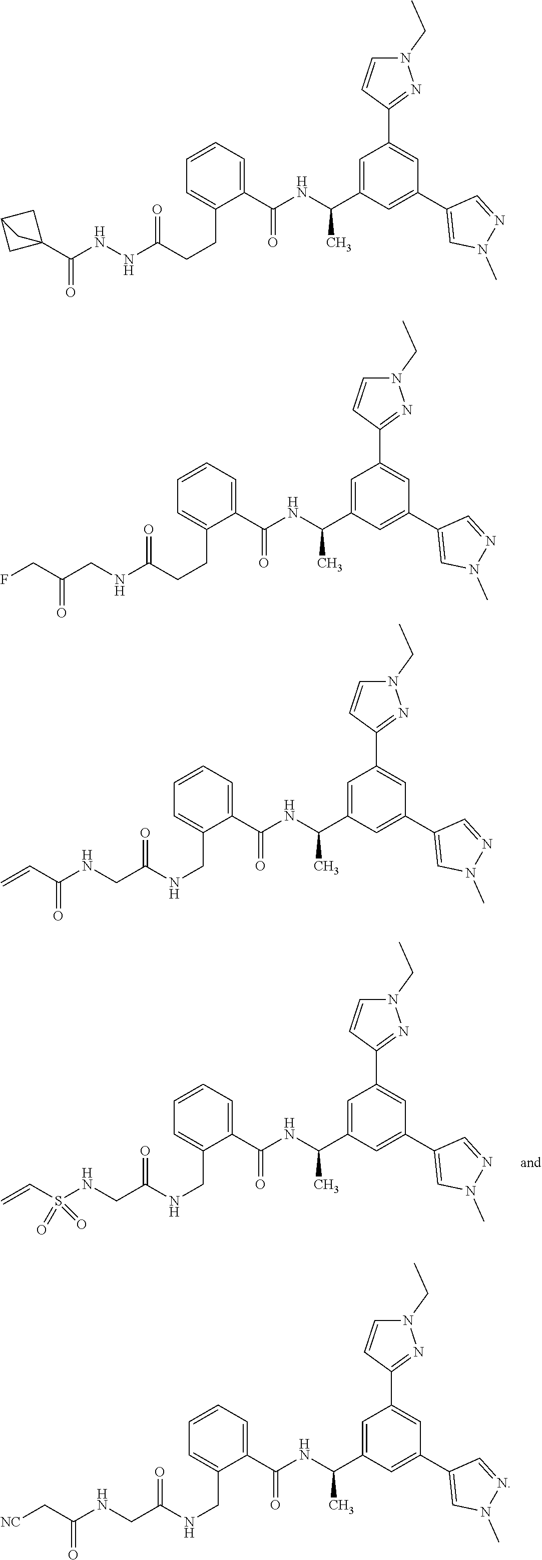 [SMILES: CCn1ccc(-c2cc(-c3cnn(C)c3)cc([C@@H](C)NC(=O)c3ccccc3CCC(=O)NNC(=O)C34CC(C3)C4)c2)n1 ; CCn1ccc(-c2cc(-c3cnn(C)c3)cc([C@@H](C)NC(=O)c3ccccc3CCC(=O)NCC(=O)CF)c2)n1 ; C=CC(=O)NCC(=O)NCc1ccccc1C(=O)N[C@H](C)c1cc(-c2cnn(C)c2)cc(-c2ccn(CC)n2)c1 ; C=CS(=O)(=O)NCC(=O)NCc1ccccc1C(=O)N[C@H](C)c1cc(-c2cnn(C)c2)cc(-c2ccn(CC)n2)c1 ; "and" ; CCn1ccc(-c2cc(-c3cnn(C)c3)cc([C@@H](C)NC(=O)c3ccccc3CNC(=O)CNC(=O)CC#N)c2)n1]
and salts thereof.

19. A pharmaceutical composition comprising a compound as described in claim 1, or a pharmaceutically acceptable salt thereof and a pharmaceutically acceptable carrier.

20. A method for promoting an antiviral effect in an animal, comprising administering a compound as described in claim 1, or a pharmaceutically acceptable salt thereof to the animal.
